(19) United States
(12) Patent Application Publication
(10) Pub. No.: US 2012/0100562 A1
(43) Pub. Date: Apr. 26, 2012

(54) METHODS AND COMPOSITIONS FOR MODULATING THE WNT PATHWAY

(75) Inventors: Eric Bourhis, San Francisco, CA (US); Andrea Cochran, San Francisco, CA (US); Yingnan Zhang, South San Francisco, CA (US)

(73) Assignee: Genentech, Inc., South San Francisco, CA (US)

(21) Appl. No.: 13/276,611

(22) Filed: Oct. 19, 2011

Related U.S. Application Data
(60) Provisional application No. 61/394,840, filed on Oct. 20, 2010.

(51) Int. Cl. G01N 33/566; C07K 7/06; C07K 7/08; C07K 7/64; C07K 5/117; C07K 5/11; C07K 5/12

(52) U.S. Cl. 435/7.92; 530/330; 530/329; 530/328; 530/321; 530/327; 530/326; 436/501

(57) ABSTRACT

The invention provides methods and compositions for modulating the Wnt signaling pathway, in particular by interfering with binding of Dkk1 or SOST with LRP5 and/or LRP6.

### CROSS REFERENCE TO RELATED APPLICATION

This application claims the benefit of U.S. Provisional Application No. 61/394,840, filed Oct. 20, 2010, the disclosure of which is incorporated herein by reference in its entirety.

### TECHNICAL FIELD

The present invention relates generally to the field of Wnt pathway regulation. More specifically, the invention concerns modulators of the Wnt signaling pathway, and uses of said modulators.

### BACKGROUND

The Wnt/β-catenin signaling pathway is essential from embryonic development to adult organism homeostasis, and if deregulated, can induce diseases ranging from osteoporosis to cancer (1-4). The first Wnt gene, originally named int-1 (5), was discovered in 1982 and later reclassified as the founding member of the Wnt gene family upon discovery of its homolog Wg in Drosophila (6, 7). Within the last three decades, proteins constituting the core of the Wnt/β-catenin signaling have been identified which define off and on states of this pathway. In the absence of Wnt ligand, intracellular β-catenin is part of a complex formed by Axin, APC, GSK3 and CK1 which phosphorylates and target β-catenin for degradation by the proteasome upon ubiquitination by β-Trcp (2). Wnt/β-catenin signaling is initiated by binding of the secreted Wnt to its co-receptors Frizzled (Fz) (8) and low density lipoprotein receptor-related protein 5 or 6 (9, 10). Wnt mediated binding of Fz to LRP induces the formation of a ternary complex at the cell surface (10, 11) which results in association of the protein Dishevelled (Dvl) with the intracellular domain of Fz and the phosphorylation of the LRP6 C-terminal PPPSPxS motif by the protein kinases GSK3 and CK1, two events necessary for the recruitment of Axin to the plasma membrane (12-15). Wnt mediated displacement of Axin induces the stabilization of the β-catenin cytoplasmic pool, and allows its translocation to the nucleus, where it acts as a co-transcriptional factor in complex with TCF/LEF to activate expression of the Wnt target genes (2).

The Wnt/β-catenin pathway has been linked to metabolic disorders (16), neurodegeneration (17, 18), and numerous types of cancers (1, 2, 4). A more established link exists between mutations of the APC protein, which prevent full β-catenin regulation, and colorectal cancers (4, 19, 20). Of particular note is the genetic relationship between LRP5 and bone homeostasis. Loss of function mutations in LRP5 cause the autosomal recessive disorder osteoporosis-pseudoglioma syndrome (OPPG), characterized by low bone mass, ocular defects and a predisposition to fractures (21). Conversely, additional genetic characterization of LRP5 revealed mutations translating in a high bone-mass density phenotype (22-24).

At the cell surface, Wnt/β-catenin signaling is regulated by two groups of secreted proteins with distinct modes of action. First, the soluble Frizzled-related protein, or sFRPs (25), have a similar fold to the cysteine-rich domain (CRD) of the Frizzled receptor (26) and inhibit the Wnt/β-catenin pathway by directly binding to the Wnt protein. A second type of Wnt-binding inhibitors, the Wnt inhibitory factor (WIF) is composed instead of a WIF domain and five EGF domains (27), which indicates that the Wnt proteins can interact with structurally different inhibitors. The second class of Wnt inhibitors is composed of the Dickkopf (Dkk) (28, 29) and WISE/Sclerostin (30-32) families of proteins. These proteins inhibit the Wnt/β-catenin signaling pathway by directly competing with Wnt for binding to its co-receptors LRP5 and LRP6 (29, 33). Both Dkk1 and Sclerostin (SOST) have been shown to be directly involved in bone growth regulation by LRP5. In particular, Sclerostin loss of function is responsible for sclerosteosis and Van Buchem diseases (34, 35); the unusually dense and strong bone observed in these conditions is similar to the hBMD phenotype caused by to LRP5 gain-of-function mutations. Dkk1 mutations causing comparable effects have not been found, even though the function of Dkk1 in murine bone development is comparable to that of Sclerostin (36).

At present, parathyroid hormone (PTH) represents the only FDA-approved bone-forming product available on the market, but PTH has been associated with safety issues such as hypercalcemia and osteosarcoma (37). Other treatments, such as biphosphonate and antibodies targeting the receptor activator of nuclear factor-κB (RANKL), target the osteoclast cell subtype which has the effect of decreasing bone resorption (38). Alternatively, the Wnt/β-catenin signaling pathway stimulates osteoblastogenesis (39) and, therefore, stimulation of Wnt signaling can induce bone formation (40). With an aging population pre-disposed to fractures, osteoporosis and rheumatoid arthritis, there is a need for safe and therapeutically effective bone anabolic agents.

### SUMMARY

The invention provides compounds that modulate the Wnt pathway and methods of using the same. One aspect of the invention provides for a compound that inhibits the binding of Dkk1 and/or SOST to LRP6 and/or LRP5. In one embodiment, the compound does not inhibit the binding of a Wnt to LRP6 and/or LRP6. In one embodiment, the compound does not inhibit binding of Wnt9B to LRP6 and/or LRP5.

One aspect of the invention provides for an isolated peptide comprising the amino acid sequence X₀X₁X₂X₃ where X₀ is N; X₁ is A, S, F, T, Y, L, or K, or R; X₂ is I or V; and X₃ is K, R, or H. In one embodiment, the peptide comprises the amino acid sequence X₁X₀X₁X₂X₃X_{4,} where X₋₁ is P, S, C, or G; X₀ is N; X₁ is A, S, F, T, Y, L, or K, or R; X₂ is I or V; X₃ is K, R, or H; _{and} X₄ is F, T, Y, L, or V. In one embodiment, the peptide comprises an amino acid sequence selected from the group consisting of N X₁IK, N X₁VK, N X₁ IR, N X₁ VR, N X₁ IH, and N X₁VH, where X₁ is A, S, F, T, Y. In one embodiment, the peptide is selected from among the peptides of Family 1 (FIG. 1). In one embodiment, at least one amino acid of the peptide is substituted with an amino acid analog. In one embodiment, the peptide comprises an amino acid analog. In one embodiment, the peptide inhibits the binding of Dkk1 to LRP6 and does not inhibit the binding of Wnt9B to LRP6. In one embodiment, the peptide binds to the E1 β-propeller of LRP6. In one embodiment, the peptide interacts with at at least one, at least two, at least three, at least four, at least five, at least six, at least seven, at least eight, at least nine, at least ten, at least eleven, or all of the amino acid residues R28, E51, D52, V70, S71, E73, L95, S96, D98, E115, R141, and N185 of the E1 β-propeller of LRP6.

One aspect of the invention provides for an isolated cyclic peptide comprising the amino acid sequence: X₀X₁X₂X_{3,} where X₀ is N; X₁ is F, Y, L, A, R, or S; X₂ is I or V; and X₃ is K, R, or H. In one embodiment, the cyclic peptide comprises the amino acid sequence X₋₁X₀X₁X₂X₃X_{4,} where X₋₁ is P, S, C, or G; X₀ is N; X₁ is F, Y, L, A, R, or S; X₂ is I or V; X₃ is K, R, or H; and X₄ is F, T, Y, L, or V. In one embodiment, the cyclic peptide comprises an amino acid sequence from the group consisting of N X₁IK, N X₁VK, N X₁ IR, N X₁ VR, N X₁ IH, and N X₁VH, where X₁ is F, Y, L, A, R, or S. In one embodiment, the cyclic peptide is selected from among the peptides of Family 2 (FIG. 2). In one embodiment, at least one amino acid of the cyclic peptide is substituted with an amino acid analog. In one embodiment, the cyclic peptide comprises an amino acid analog. In one embodiment, the cyclic peptide inhibits the binding of Dkk1 to LRP6 and does not inhibit the binding of Wnt9B to LRP6. In one embodiment, the cyclic peptide binds to the E1 β-propeller of LRP6. In one embodiment, the cyclic peptide interacts with at at least one, at least two, at least three, at least four, at least five, at least six, at least seven, at least eight, at least nine, at least ten, at least eleven, or all of the amino acid residues R28, E51, D52, V70, S71, E73, L95, S96, D98, E115, R141, and N185 of the E1 β-propeller of LRP6.

One aspect of the invention provides for an isolated peptide comprising the amino acid sequence: X₋₁X₀X₁X_{2,} where X₋₁ is W, L, Y, F, or I; X₀ is D or E; X₁ is F, W, I, S, or Y; and X₂ is M. In one embodiment, the peptide comprises the amino acid sequence: X₋₂X₋₁X₀X₁X₂X_{3,} where X₋₂ is V, I, L, or F; X₋₁ is W, L, Y, F, or I; X₀ is D or E; X₁ is F, W, I, S, or Y; X₂ is M; and X₃ is W, M, A, or G. In one embodiment, the peptide is selected from among the peptides of Family 3 (FIG. 3).

One aspect of the invention provides for an isolated peptide selected from among the peptides of Family 4 (FIG. 4).

One aspect of the invention provides for a method for screening for a compound that inhibits the interaction of Dkk1 and LRP6 comprising contacting a test compound with LRP6, or functional equivalent thereof, and determining the level of binding of the test compound to the LRP6, or functional equivalent thereof, in the presence and the absence of a peptide ligand that inhibits the interaction of Dkk1 with LRP6 wherein a change in level of binding in the presence or absence of the peptide ligand indicates that the test compound inhibits the interaction of Dkk1 with LRP6 and wherein the peptide ligand comprises an amino acid sequence selected from the group consisting of the amino acid sequences of a) Family 1 (FIG. 1); b) Family 2 (FIG. 2); c) Family 3 (FIG. 3); and d) Family 4 (FIG. 4). In one embodiment, the peptide ligand is labeled with a detectable label.

One aspect of the invention provides for a method for screening for a compound that inhibits the interaction of Dkk1 and LRP5 comprising contacting a test compound with LRP5, or functional equivalent thereof, and determining the level of binding of the test compound to the LRP5, or functional equivalent thereof, in the presence and the absence of a peptide ligand that inhibits the interaction of Dkk1 with LRP5 wherein a change in level of binding in the presence or absence of the peptide ligand indicates that the test compound inhibits the interaction of Dkk1 with LRP5 and wherein the peptide ligand comprises an amino acid sequence selected from the group consisting of the amino acid sequences of a) Family 1 (FIG. 1); b) Family 2 (FIG. 2); c) Family 3 (FIG. 3); and d) Family 4 (FIG. 4). In one embodiment, the peptide ligand is labeled with a detectable label.

### BRIEF DESCRIPTION OF THE DRAWINGS

FIG. 1. Exemplary peptides of Family 1.

FIG. 2. Exemplary peptides of Family 2.

FIG. 3. Exemplary peptides of Family 3.

FIG. 4A-C. Exemplary peptides of Family 4.

FIG. 5. Detailed view of the CDR H3 interaction with residues of the LRP6 groove showing the network of interactions made by the NAVK sequence.

FIG. 6. Detail of the interactions made by antibody CDRs other than H3.

FIG. 7. (A) Alignment of primary sequences from Dkk1, Dkk2, Dkk4, Sclerostin, and Wise. (B) Examples of peptides based on proteins with “NXI” motif.

FIG. 8. Competition binding between Dkk1 and other Wnt pathway inhibitors. The indicated LRP6 construct was preloaded onto biosensor tips. Dkk1 (100 nM) (or buffer control) and the test ligand (100 nM) were loaded sequentially onto the LRP6 tips. (A) Dkk2 competition with Dkk1. (B) Sclerostin competition with Dkk1. Percent binding in the presence of Dkk1 is shown relative to buffer control.

FIG. 9. Binding determinants in the Wnt inhibitors Dkk1 and sclerostin (A) The conserved Asn and Ile residues of the “NXI” motif are important for Dkk1 and sclerostin binding to LRP6 E1E2. (B) Dkk1 has two independent binding regions, one that recognizes LRP6 E1E2 and one that recognizes LRP6 E3E4. Substitutions in the “NXI” motif (N40A, I42E) affect binding to LRP6 E1E2 but not to E3E4, whereas substitutions in the C-terminal cysteine-rich domain (H204E, K211E) affect binding to LRP6 E3E4 but not to E1E2. In each case, mutant proteins retain binding to LRP6 E1E4.

FIG. 10. Cartoon depicting the different Dkk1-LRP6 E1E4 complexes studied by SEC-MALS and possible models for the interaction. Predicted molecular weights for each individual molecule or complex are indicated, with experimentally observed weights shown below. The observed molecular weights are consistent with 1:1 complex formation between LRP6 E1E4 and each of the Dkk1 variants. The data are not consistent with model 3 (showing a 2:1 stoichiometry). The data are instead consistent with either model 4, in which one Dkk1 molecule can bridge two LRP6 binding sites, or model 5/6, in which only one or the other site is accessible to bound Dkk1.

FIG. 11. Wnt binding to LRP6 E1E4 in the presence or absence of Dkk1 or sclerostin. Dkk1 (125 nM) inhibits binding of both Wnt3A and Wnt9B (125 nM each), while sclerostin (125 nM) only inhibits binding of Wnt9B.

FIG. 12. Induction of a Wnt/β-catenin reporter in the presence or absence of wild-type and mutant inhibitors. Cells were transfected by Wnt1 (binds to LRP6 E1E2). Dkk1 and sclerostin variants, or the control inhibitor Fz8 CRD, were used at the indicated doses.

FIG. 13. Introduction of LRP5 BMD substitutions into LRP6 E1E2 lowers affinity for Wnt inhibitors. The five substitutions characterized are indicated on the y-axis. Steady-state affinity measurements were made for Wnt9b, Dkk1, and sclerostin binding to each of the LRP6 variants. Differences in binding to Wnt9b were minor (≦5-fold change compared to wild type), while binding to Dkk1 and sclerostin was more significantly impacted (10-250-fold losses in affinity compared to wild type).

FIG. 14. Conserved motifs present in phage clones selected from linear and cyclic peptide libraries against LRP6 E1E2 (A) Linear peptides of Exemplary Family 1. (B) Cyclic peptides of Exemplary Family 2.

FIG. 15. Conserved motifs present in phage clones selected from linear and cyclic peptide libraries against LRP5 E1 (A) Linear peptides of Exemplary Family 3. (B) Cyclic peptides of Exemplary Family 4.

FIG. 16. Co-crystal structures of LRP6 E1 and peptides discovered from phage-display libraries. (A) Peptide Ac-SNSIKFYA-am from Exemplary Family 1. (B) Peptide Ac-GSLCSNRIKPDTHCSS-am (disulfide), a CX₉C class member of Exemplary Family 2. (C) Peptide Ac-CNSIKLC-am (disulfide), a CX₅C class member of Exemplary Family 2. (D) Peptide Ac-CNSIKCL-am (disulfide), a CX₄C class member of Exemplary Family 2.

FIG. 17. Structure-activity study of the Dkk1 7-mer peptide. The indicated peptides were synthesized by standard Fmoc procedures, and IC₅₀ values were determined as described in Example 1. (A) C-terminal and N-terminal truncations. (B) Substitutions at position “X” of the “NXI” motif.

FIGS. 18A and B. Structure-activity study of the Dkk1 7-mer peptide showing effects of substitution of the N, S, I, and K residues. The indicated peptides were synthesized by standard Fmoc procedures, and IC₅₀ values were determined as described in Example 1.

FIG. 19. Structure-activity study of a linear peptide from Exemplary Family 1. Substitutions were made in the Ile position of the “NXI” motif. The indicated peptides were synthesized by standard Fmoc procedures, and IC₅₀ values were determined as described in Example 1.

FIG. 20. Transfer of the “NXI” epitope to a structured peptide scaffold. (A) Design of the structured mimetic. The residues N100-V100b from the antibody complex structure were overlaid on a representative structure of a Bowmain-Birk inhibitory (BBI) loop peptide (PDB code 1 GM2) (42). Apart from an amide bond rotation preceding the branched hydrophobic residue, the conformations of the peptides are similar. The positions of side chain β-carbons for the three-residue motif coincide. Sequences of the BBI loop template and the “NXI”-containing BBI mimetic are shown. (B) The BBI mimetic binds to LRP6 E1, while a control peptide lacking the conserved Asn does not.

FIG. 21. Design of a amide-cyclized variant of the Dkk1 7-mer peptide. (A) Structure of the Dkk1 peptide taken from the complex with LRP6 E1 is shown at top. The side chain of Ser2 points toward the side chain of Asn7 with a short gap between. Below is a model in which Ser2 is substituted by Lys, and Asn7 by Asp. The side chains are joined by an amide bond between the Lys c-amine and the Asp carboxylate. (B) Competition binding data indicate that the cyclized peptide binds to LRP6 E1.

FIG. 22. LRP6 E1-binding peptides inhibit binding of Wnt inhibitors, but not of Wnt9B, to LRP6 E1E2. Binding was assessed by biolayer interferometry, as described in Example 1. Immobilized LRP6 E1E2 was exposed to protein ligand (Wnt 9b, Dkk1, or sclerostin) present in solution at a concentration three-fold higher than the measured dissociation constant for E1E2. Competing peptides were added at a saturating level (20-fold higher than the measured IC₅₀ value). Peptide A: Ac-NSNAIKN-am; Peptide B: Ac-CNSIKFCG-am (disulfide); Peptide C: Ac-GSLCSNRIKPDTHCSS-am (disulfide)

### DISCLOSURE OF THE INVENTION

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988).

Definitions

The term “amino acid” within the scope of the present invention is used in its broadest sense and is meant to include the naturally- occurring L -amino acids or residues. The commonly used one- and three-letter abbreviations for naturally-occurring amino acids are used herein (Lehninger, Biochemistry, 2d ed., pp. 71-92, (Worth Publishers: New York, 1975). The term includes D-amino acids as well as chemically-modified amino acids such as amino acid analogs, naturally-occurring amino acids that are not usually incorporated into proteins such as norleucine, and chemically-synthesized compounds having properties known in the art to be characteristic of an amino acid. For example, analogs or mimetics of phenylalanine or proline, which allow the same conformational restriction of the peptide compounds as natural Phe or Pro, are included within the definition of amino acid. Such analogs and mimetics are referred to herein as “functional equivalents” of an amino acid. Other examples of amino acids are listed by Roberts and Vellaccio, The Peptides: Analysis, Synthesis, Biology, Eds. Gross and Meiehofer, Vol. 5, p. 341 (Academic Press, Inc.: N.Y. 1983).

In certain embodiments, variants of compounds, such as peptide variants having one or more amino acid substitutions, are provided. Conservative substitutions are shown in Table 1 under the heading of “conservative substitutions.” More substantial changes are provided in Table 1 under the heading of “exemplary substitutions,” and as further described below in reference to amino acid side chain classes.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

Synthetic peptides, synthesized for example by standard solid-phase synthesis techniques, are not limited to amino acids encoded by genes and therefore allow a wider variety of substitutions for a given amino acid Amino acids that are not encoded by the genetic code are referred to herein as “amino acid analogs” and include, for example, those described in WO 90/01940 and in the table below (Table 2), as well as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu, and other aliphatic amino acids; 2-aminoisobutyric acid (Aib) for Gly; cyclohexylalanine (Cha) for Val, Leu and Ile; homoarginine (Har) for Arg and Lys; 2,3-diaminopropionic acid (Dap) for Lys, Arg, and His; N-ethylglycine (EtGly) for Gly, Pro, and Ala; N-ethylglycine (EtGly) for Gly, Pro, and Ala; N-ethylasparagine (EtAsn) for Asn, and Gln; hydroxylysine (Hyl) for Lys; allohydroxylysine (AHyl) for Lys; 3-(and 4-)hydroxyproline (3Hyp, 4Hyp) for Pro, Ser, and Thr; allo-isoleucine (AIle) for Ile, Leu, and Val; 4-amidinophenylalanine for Arg; N-methylglycine (MeGly, sarcosine) for Gly, Pro, and Ala; N-methylisoleucine (Mae) for Ile; norvaline (Nva) for Met and other aliphatic amino acids; norleucine (Nle) for Met and other aliphatic amino acids; ornithine (Orn) for Lys, Arg and His; citrulline (Cit) and methionine sulfoxide (MSO) for Thr, Asn, and Gln; and N-methylphenylalanine (MePhe), trimethylphenylalanine, halo-(F-, Cl-, Br-, or I-)phenylalanine, or trifluorylphenylalanine for Phe.

TABLE 2
Examples of hydrophobic amino acid analogs that may
be incorporated into the peptides of the invention¹

| Name | Common abbreviation |
|---|---|
| Cyclohexylglycine | Chg |
| Cyclopentylglycine | Cpg |
| Cyclobutylalanine | |
| Cyclopropylalanine | |
| tert-Leucine | Tle |
| Norleucine | Nle |
| Norvaline | Nva |
| 2-Aminobutyric acid | Abu |

¹Non-genetically encoded amino acids corresponding to those used in Example 13. This list is not meant to be exhaustive and other substitutions may be contemplated.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Unless specifically stated otherwise, all % amino acid sequence identity values used herein are obtained as described in the immediately preceding paragraph using the ALIGN-2 computer program.

An “isolated” compound is one which has been separated from a component of its natural environment. In some embodiments, a compound, such as a peptide, is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The term “LRP6”, as used herein, refers to any native LRP6 from any vertebrate source, including mammals such as primates (e.g. humans) and rodents (e.g., mice and rats), unless otherwise indicated. The term encompasses “full-length,” unprocessed LRP6 as well as any form of LRP6 that results from processing in the cell. The term also encompasses naturally occurring variants of LRP6, e.g., splice variants or allelic variants. The amino acid sequence of an exemplary human LRP6 is provided in NCBI accession number AAI43726, Strausberg, R. L., et al., Proc. Natl. Acad. Sci. U.S.A. 99: 16899-16903 (2002) (He, X, et al., Development, 131:1663-1677 (2004); Chen, M., et al., J. Biol. Chem., 284:35040-35048 (2009).

The term “LRP5”, as used herein, refers to any native LRP5 from any vertebrate source, including mammals such as primates (e.g. humans) and rodents (e.g., mice and rats), unless otherwise indicated. The term encompasses “full-length,” unprocessed LRP5 as well as any form of LRP5 that results from processing in the cell. The term also encompasses naturally occurring variants of LRP5, e.g., splice variants or allelic variants. The amino acid sequence of an exemplary human LRP5 is provided in NCBI accession number O75197, Hey, P. J., et al, Gene 216 (1), 103-111 (1998).

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compounds of the invention are used to delay development of a disease or to slow the progression of a disease.

The terms “antibody” and “immunoglobulin” are used interchangeably in the broadest sense and include monoclonal antibodies (for e.g., full length or intact monoclonal antibodies), polyclonal antibodies, multivalent antibodies, multispecific antibodies (e.g., bispecific antibodies so long as they exhibit the desired biological activity) and may also include certain antibody fragments (as described in greater detail herein). An antibody can be human, humanized and/or affinity matured.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “disorder” is any condition that would benefit from treatment with a substance/molecule or method of the invention. This includes chronic and acute disorders or diseases including those pathological conditions which predispose the mammal to the disorder in question. Non-limiting examples of disorders to be treated herein include disorders of processes that are activated or inhibited by Wnt signaling. Such processes include, for example, cell proliferation, cell fate specification, and stem cell self-renewal in different cancer types, and developmental processes. The compounds of the invention are useful, for example, in the treatment of Wnt mediated disorders of the bones or skeletal system. Examples of skeletal or bone disorders that can be treated using the compounds of the invention include osteoporosis, osteoarthritis, bone fractures, and bone lesions and various forms of bone degeneration.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

An “effective amount” refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, R¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

Compounds and Methods

The Dickkopf (Dkk) and WISE/Sclerostin (SOST) family of proteins inhibit the Wnt/β-catenin signaling pathway by directly competing with Wnt for binding to its LRP5 and LRP6 co-receptors. Provided herein are compounds that modulate the interaction of DKK1 with LRP5 and/or LRP6 and compounds that modulate the interaction of SOST with LRP5 and/or LRP6. In some embodiments, a compound modulates the interactions of both Dkk1 and SOST with LRP5/and or LRP6.

In one embodiment, the compound inhibits the interaction of Dkk1 with LRP5 and/or LRP6. In one embodiment, the compound inhibits the interaction of SOST with LRP5 and/or LRP6. In one embodiment, the compound inhibits the interactions of both Dkk1 and SOST with LRP5 and/or LRP6.

In one embodiment, the compound competes for binding to LRP6 with Dkk1. In one embodiment, the compound competes for binding to LRP6 with SOST. In one embodiment, the compound competes for binding to LRP5 with Dkk1. In one embodiment, the compound competes for binding to LRP5 with SOST. In one embodiment, the compound binds to a Dkk1 binding site on LRP6. In one embodiment, the compound binds to a SOST binding site on LRP6. In one embodiment, the compound binds to a Dkk1 binding site on LRP5. In one embodiment, the compound binds to a SOST binding site on LRP5. In one embodiment, the compound binds to the E1 β-propeller of LRP6. In one embodiment, the compound binds to the E1 β-propeller of LRP5. In one embodiment, the compound interacts with at least one, at least two, at least three, at least four, at least five, at least six, at least seven, at least eight, at least nine, at least ten, at least eleven, or all of the amino acid residues R28, E51, D52, V70, S71, E73, L95, S96, D98, E115, R141, and N185 of the E1 β-propeller of LRP6. In one embodiment, the compound interacts with at least one, at least two, at least three, at least four, at least five, at least six, at least seven, at least eight, at least nine, at least ten, at least eleven, or all of the amino acid residues R28, E63, D64, V82, S83, E85, V108, S109, D111, E128, R154, and N198 of the E1 β-propeller of LRP5. By directly binding to the Dkk1 or SOST binding site, the compound provides a targeted approach to modulating the Wnt pathway signaling associated with binding of Dkk1 and SOST. In one embodiment, the compound modulates Wnt pathway signaling associated with binding of Dkk1 to LRP5 or LRP6. In one embodiment, the compound modulates Wnt pathway signaling associated with binding of SOST to LRP5 or LRP6. In one embodiment, the compound modulates the Wnt pathway signaling associated with binding of Dkk1 and/or SOST to LRP5 or LRP6 without modulating the serotonin pathway.

In some embodiments, the compound inhibits the interaction of Dkk1 with LRP5 and/or LRP6 and does not inhibit the interaction of a Wnt with LRP5 or LRP6. In some embodiments, the compound inhibits the interaction of SOST with LRP5 and/or LRP6 and does not inhibit the interaction of a Wnt with LRP5 or LRP6. In one embodiment, the Wnt is Wnt3a. In one embodiment, the Wnt is Wnt9b. This selective inhibition serves to prevent inhibition of the Wnt signaling pathway by the inhibitors Dkk1 or SOST while allowing for the stimulation of the pathway by Wnt molecules. As a result, the compounds serve to promote bone growth and repair associated with the Wnt pathway.

In some embodiments, the compounds find use in the treatment of various skeletal disorders that can benefit from the promotion of bone growth such as, for example, osteoporosis, rheumatoid arthritis, bone degradation or degeneration which can occur due to a number of conditions including, for example, cancers such as multiple myeloma, and in the treatment of bone fractures or other bone deficiencies associated with low bone density or low bone strength.

In one aspect of the invention, the compound is a peptide. In one embodiment, the compound is a linear peptide. In embodiment, the linear peptide is from 3 to 100, 3 to 50, 3 to 30, 3 to 20, 3 to 10, 3 to 9, 3 to 8, 3 to 7, 3 to 6, 3 to 5, or 3 to 4 amino acids in length. In one embodiment, the linear peptide is from 4 to 10, 5 to 8, 6 to 7 amino acids in length. In one embodiment, the linear peptide is 3, 4, 5, 6, 7, 8, 9, or 10 amino acids in length. In another embodiment, the compound is a cyclic peptide. In embodiment, the cyclic peptide is from 5 to 100, 5 to 50, 5 to 30, 5 to 20, 5 to 10, 7 to 20. 7 to 17, 7 to 16, 7 to 17, 7 to 18, 7 to 19, or 7 to 20 amino acids in length. In one embodiment, the cyclic peptide is 5, 6, 7, 8, 9, 10, 11, 12, 13, 14, 15, 16, 17, 18, 19, or 20 amino acids in length.

In a further embodiment, the peptide is a structured peptide or a peptide that adopts a well-defined conformation in the absence of binding to the target (adoptive peptide). This conformation adopted by the peptide is similar to the conformation of the bound-state structure of the peptide. In some embodiments, the structured peptide or adoptive peptide has enhanced therapeutic efficacy as compared to an unstructured peptide. In one embodiment, the structured peptide or adoptive peptide has one or more of the characteristics of enhanced target binding, enhanced stability, and enhanced bioavailability as compared to an unstructured peptide.

In one aspect, the invention provides a linear peptide of Family 1 comprising the amino acid sequence: X₀X₁X₂X₃ where X₀ is an asparagine (N) residue. The peptides of Family 1 bind to the E1 β-propeller of LRP6. In some embodiments, peptides of Family 1 also bind to LRP5. In one embodiment, X₀ is N; X₁ is A, S, F, T, Y, L, K or R; X₂ is I or V; and X₃ is K, R, or H. In one embodiment, X₀ is N; X₁ is A, S, F, T, Y, L, K, or R; X₂ is I; and X₃ is K, R, or H. In one embodiment, X₀ is N; X₁ is A, S, F, T, Y, L, K, or R; X₂ is I or V; and X₃ is K. In one embodiment, X₀ is N; X₁ is A, S, F, T, Y, L, K, or R; X₂ is V; and X₃ is K, R, or H. In one embodiment, X₀ is N; X₁ is A, S, F, T, Y, L, K, or R; X₂ is I; and X₃ is K. In one embodiment, X₀ is N; X₁ is A, S, F, T, Y, L, K, or R; X₂ is I; and X₃ is R. In one embodiment, X₀ is N; X₁ is A, S, F, T, Y, L, K, or R; X₂ is V; and X₃ is K. In one embodiment, X₀ is N; X₁ is A, S, F, T, Y, L, K, or R; X₂ is V; and X₃ is R, or H.

In other embodiments, the linear peptide of Family 1 further comprises additional amino acid residues on either side of X₀X₁X₂X₃. In one embodiment, the invention provides for a peptide of Family 1 comprising the amino acid sequence: X₋₁X₀X₁X₂X₃X_{4,} where X₀ is N. In one embodiment, X₋₁ is P, S, C, or G; X₀ is N; X₁ is A, S, F, T, Y, L, K, or R; X₂ is I or V; X₃ is K, R, or H; _{and} X₄ X is F, T, Y, L, or V. In one embodiment, X₋₁ is P, S, C, or G; X₀ is N; X₁ is A, S, F, T, Y, L, K, or R; X₂ is I; X₃ is K, R, or H; and X₄ is F, T, Y, L, or V. In one embodiment, X₋₁ is P, S, C, or G; X₀ is N; X₁ is A, S, F, T, Y, L, K, or R; X₂ is I or V; X₃ is K; and X₄ is F, T, Y, L, or V. In one embodiment, the invention provides for a peptide of Family 1 comprising the amino acid sequence: X₋₁X₀X₁X₂X₃X₄X_{5,} where X₀ is N. In one embodiment, X₋₁ is P, S, C, or G; X₀ is N; X₁ is A, S, F, T, Y, L, K, or R; X₂ is I or V; X₃ is K, R, or H; X₄ is F, T, Y, L, or V; and X₅ is F, T, Y, L, or V. In one embodiment, X₋₁ is P, S, C, or G; X₀ is N; X₁ is A, S, F, T, Y, L, K, or R; X₂ is I; X₃ is K, R, or H; X₄ is F, T, Y, L, or V; and X₅ is F, T, Y, L, or V. In one embodiment, X₋₁ is P, S, C, or G; X₀ is N; X₁ is A, S, F, T, Y, L, K, or R; X₂ is I or V; X₃ is K; X₄ is F, T, Y, L, or V; and X₅ is F, T, Y, L, or V.

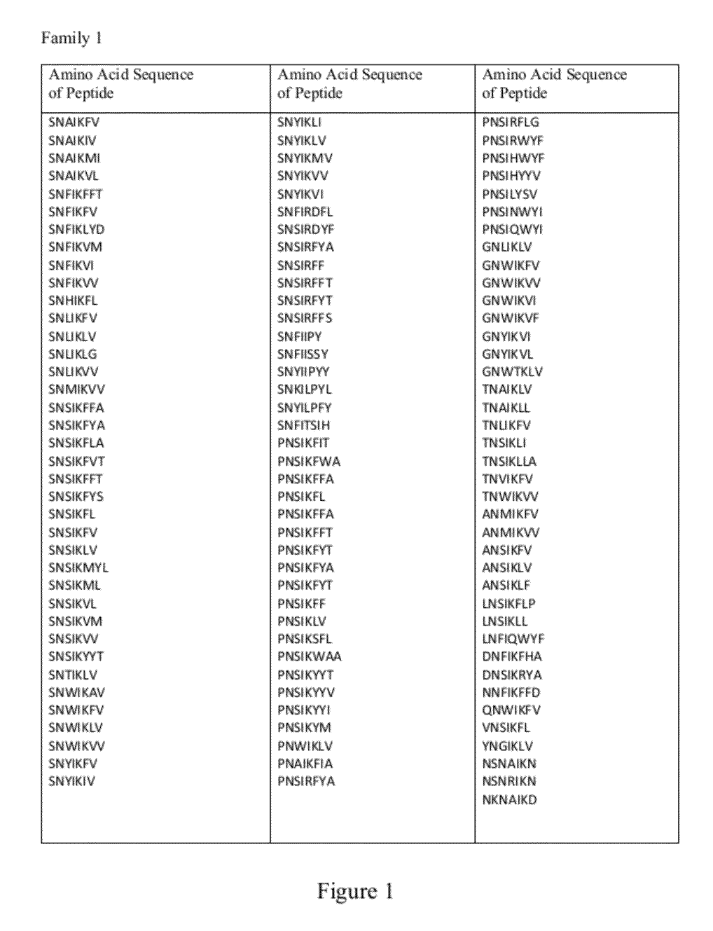

In one embodiment, the peptide of Family 1 comprises a peptide selected from the group consisting of N X₁IK, N X₁VK, N X₁ IR, N X₁ VR, N X₁ IH, and N X₁VH, where X₁ is A, S, F, T, Y, R, or K. Exemplary peptides of Family 1 are shown in FIG. 1.

In another aspect, the invention provides a cyclic peptide of Family 2 comprising the amino acid sequence: X₀X₁X₂X_{3,} where X₀ is N. The peptides of Family 2 bind to the E1 β-propeller of LRP6. In some embodiments, peptides of Family 2 also bind to LRP5. In one embodiment, X₀ is N; X₁ is F, Y, L, A, R, or S; X₂ is I or V; and X₃ is K, R, or H. In one embodiment, X₀ is N; X₁ is F, Y, L, A, R, or S; X₂ is I; and X₃ is K, R, or H. In one embodiment, X₀ is N; X₁ is F, Y, L, A, R, or S; X₂ is I or V; and X₃ is K; X₄ is F, T, Y, L, or V. In one embodiment, X₀ is N; X₁ is F, Y, L, A, R, or S; X₂ is I; and X₃ is K. In one embodiment, X₀ is N; X₁ is F, Y, L, A, R, or S; X₂ is I; and X₃ is R. In one embodiment, X₀ is N; X₁ is F, Y, L, A, R, or S; X₂ is V; and X₃ is K. In one embodiment, X₀ is N; X₁ is F, Y, L, A, R, or S; X₂ is V; and X₃ is R.

In other embodiments, the cyclic peptide of Family 2 further comprises additional amino acid residues on either side of X₀X₁X₂X_{3.} In one embodiment, the invention provides a cyclic peptide of Family 2 comprising the amino acid sequence: X₋₁X₀X₁X₂X₃X_{4,} where X₀ is N. In one embodiment, X₋₁ is P, S, C, or G; X₀ is N; X₁ is F, Y, L, A, R, or S; X₂ is I or V; X₃ is K, R, or H; and X₄ is F, T, Y, L, or V. In one embodiment, X₋₁ is P, S, C, or G; X₀ is N; X₁ is F, Y, L, A, R, or S; X₂ is I; X₃ is K, R, or H; and X₄ is F, T, Y, L, or V. In one embodiment, X₋₁ is P, S, C, or G; X₀ is N; X₁ is F, Y, L, A, R, or S; X₂ is I or V; X₃ is K; and X₄ is F, T, Y, L, or V. In another embodiment, the invention provides for a peptide of Family 1 comprising the amino acid sequence: X₁X₀X₁X₂X₃X₄X_{5,} where X₀ is N. In one embodiment, X₋₁ is P, S, C, or G; X₀ is N; X₁ is F, Y, L, A, R, or S; X₂ is I or V; X₃ is K, R, or H; X₄ is F, T, Y, L, or V; and X₅ is F, T, Y, L, or V. In one embodiment, X₋₁ is P, S, C, or G; X₀ is N; X₁ is F, Y, L, A, R, or S; X₂ is I; X₃ is K, R, or H; X₄ is F, T, Y, L, or V; and X₅ is F, T, Y, L, or V. In one embodiment, X₋₁ is P, S, C, or G; X₀ is N; X₁ is F, Y, L, A, R, or S; X₂ is I or V; X₃ is K; X₄ is F, T, Y, L, or V; and X₅ is F, T, Y, L, or V.

In one embodiment, the peptide of Family 2 comprises a peptide selected from the group consisting of N X₁IK, N X₁VK, N X₁ IR, N X₁ VR, N X₁ IH, and N X₁VH, where X₁ is F, Y, L, A, R, or S.

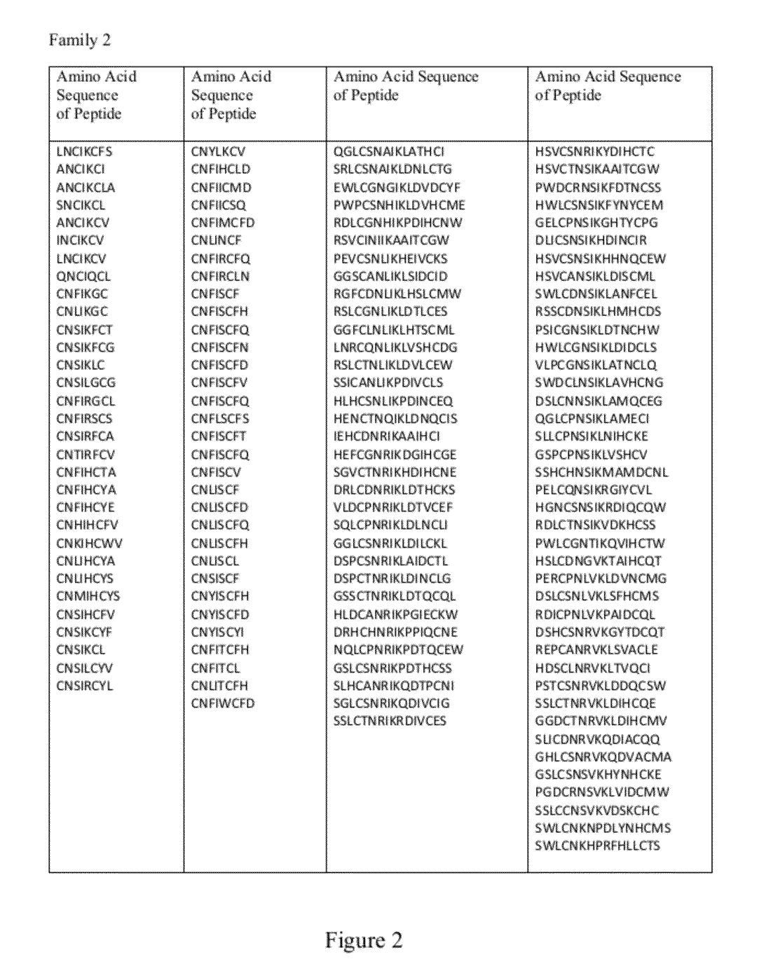

Exemplary peptides of Family 2 are shown in FIG. 2.

In another aspect, the invention provides a linear peptide of Family 3 comprising the amino acid sequence: X₋₁X₀X₁X_{2,} where X₀ is D or E and X₂ is M. The peptides of Family 3 bind to the E1 β-propeller of LRP5. In some embodiments, X₋₁ is W, L, Y, F, or I; X₀ is D or E; X₁ is F, W, I, S, or Y; and X₂ is M. In one embodiment, X₋₁ is W, L, Y, F, or I; X₀ is D; X₁ is F, W, I, S, or Y; and X₂ is M. In one embodiment, X₋₁ is W, L, Y, F, or I; X₀ is E; X₁ is F, W, I, S, or Y; X₂ is M; and X₃ is W, M, A, or G. In one embodiment, X₋₁ is F; X₀ is E; X₁ is I; X₂ is M; and X₃ is W.

In other embodiments, the linear peptide of Family 3 further comprises additional amino acid residues on either side of X₋₁X₀X₁X₂. In one embodiment, the linear peptide of Family 3 comprises the amino acid sequence: X₋₂X₋₁X₀X₁X₂X_{3,} where X₀ is D or E and X₂ is M. In one embodiment, X₋₂ is V, I, L, or F; X₋₁ is W, L, Y, F, or I; X₀ is D or E; X₁ is F, W, I, S, or Y; X₂ is M; and X₃ is W, M, A, or G. In one embodiment, X₋₂ is V, I, L, or F; X₋₁ is W, L, Y, F, or I; X₀ is D; X₁ is F, W, I, S, or Y; X₂ is M; and X₃ is W, M, A, or G. In one embodiment, X₋₂ is V, I, L, or F; X₋₁ is W, L, Y, F, or I; X₀ is E; X₁ is F, W, I, S, or Y; X₂ is M; and X₃ is W, M, A, or G. In one embodiment, X₋₂ is V; X₋₁ is F; X₀ is E; X₁ is I; X₂ is M; and X₃ is W. In another embodiment, the invention provides a linear peptide of Family 3 comprising the amino acid sequence: X₋₃X₋₂X₋₁X₀X₁X₂X_{3,} where X₀ is D or E and X₂ is M. In one embodiment, X₋₃ is H, F, N, or Q; X₋₂ is V, I, L, or F; X₋₁ is W, L, Y, F, or I; X₀ is D or E; X₁ is F, W, I, S, or Y; X₂ is M; and X₃ is W, M, A, or G. In one embodiment, X₋₃ is H, F, N, or Q; X₋₂ is V, I, L, or F; X₋₁ is W, L, Y, F, or I; X₀ is D; X₁ is F, W, I, S, or Y; X₂ is M; and X₃ is W, M, A, or G. In one embodiment, X₋₃ is H, F, N, or Q; X₋₂ is V, I, L, or F; X₋₁ is W, L, Y, F, or I; X₀ is E; X₁ is F, W, I, S, or Y; X₂ is M; and X₃ is W, M, A, or G. In one embodiment, X₋₃ is H; X₋₂ is V; X₋₁ is F; X₀ is E; X₁ is I; X₂ is M; and X₃ is W.

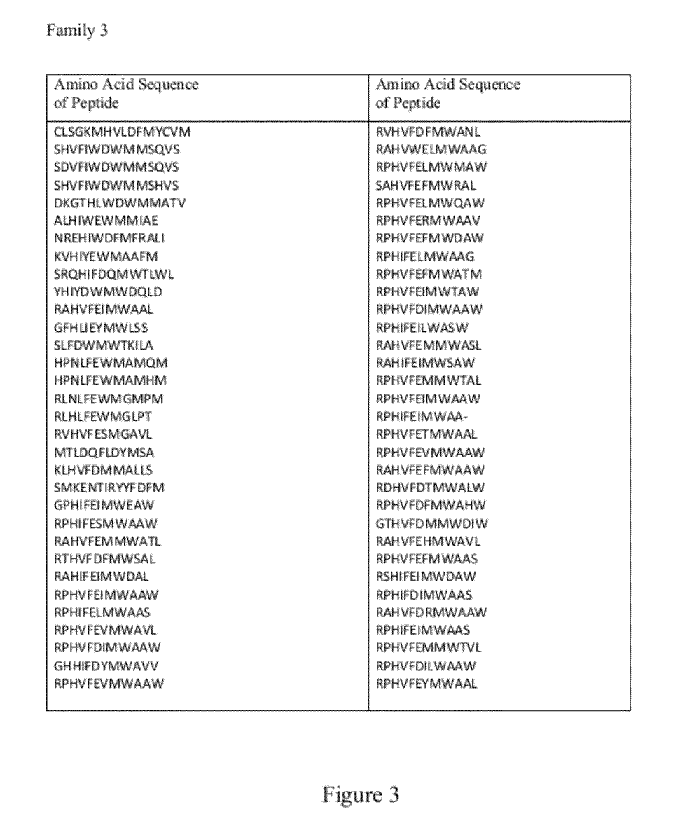

Exemplary peptides of Family 3 are shown in FIG. 3.

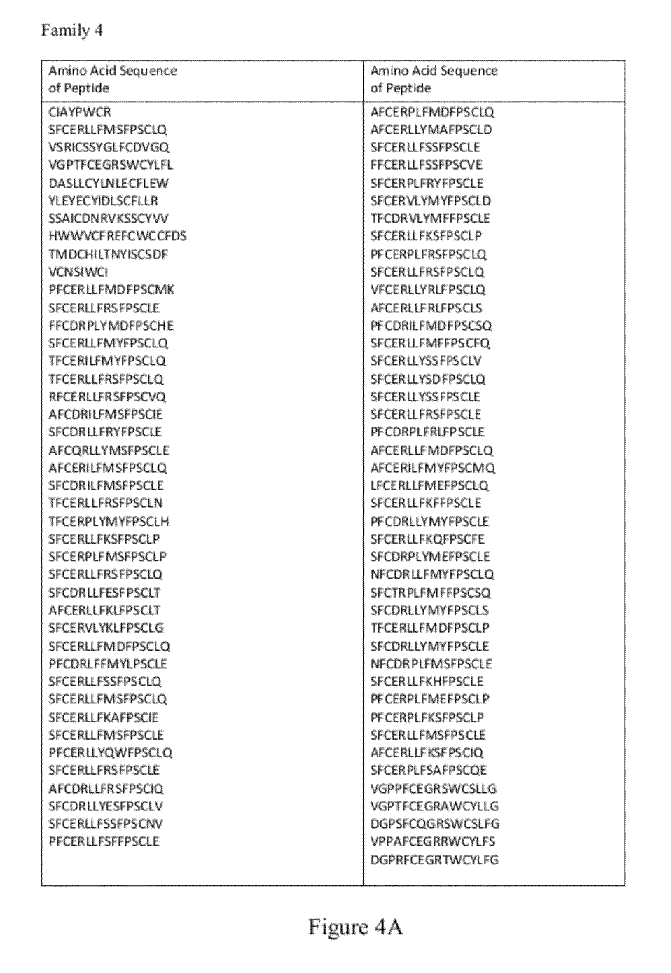

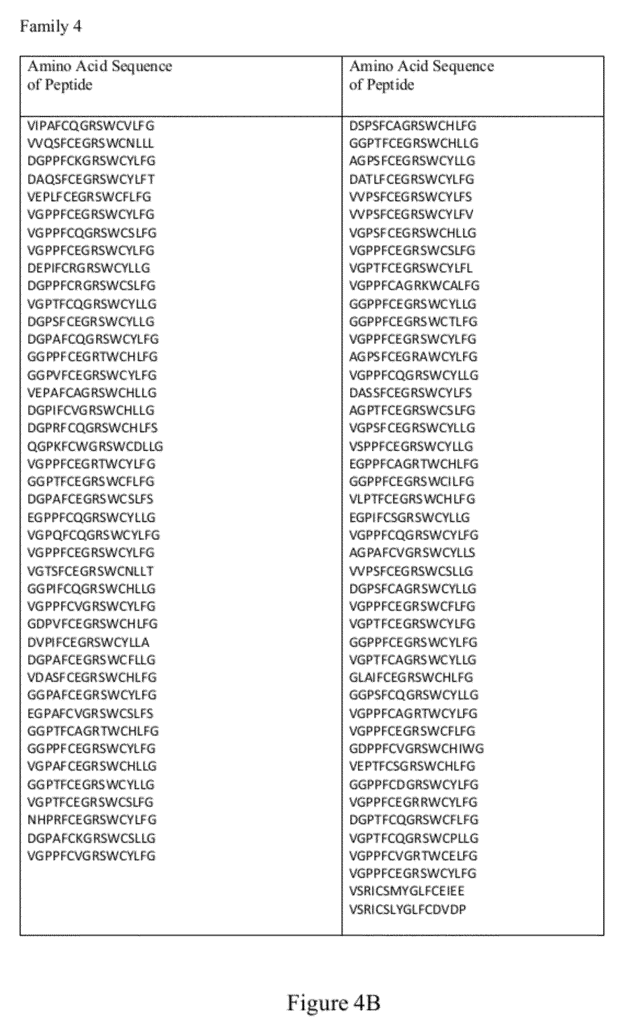

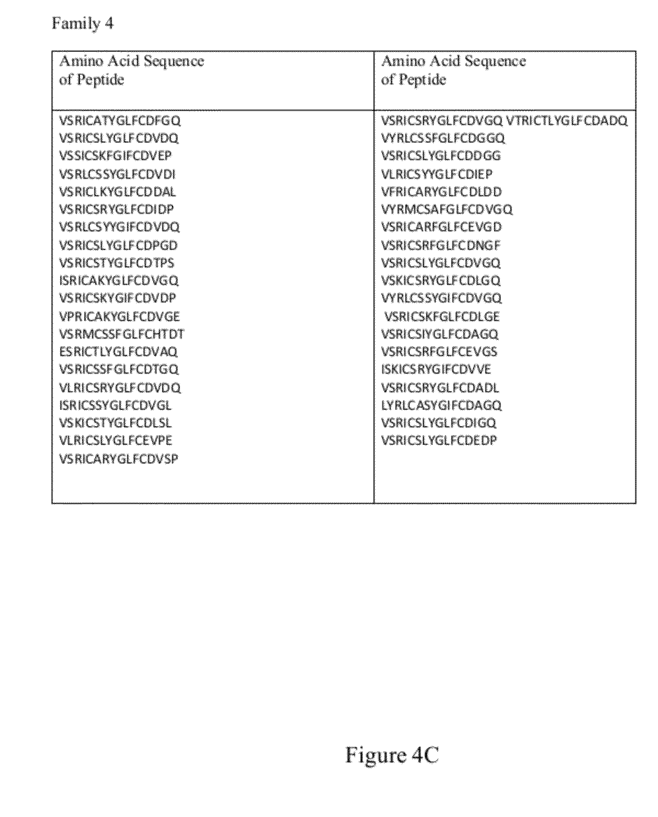

In another aspect, the invention provides a cyclic peptide of Family 4. The peptides of Family 4 bind to the E1 β-propeller of LRP5. In some embodiments, the invention provides a peptide of Family 4 as shown in FIG. 4.

In some embodiments, the peptides of the invention bind their target with a Kd of less than 100 uM, less than 50 uM, less than 20 uM, less than 10 uM, less than 5 uM, less than 1 uM, less than 0.5 uM, less than 0.1 uM, or less than 0.01 uM. In some embodiments, the peptides of the invention bind their target with a IC50 of less than 100 uM, less than 50 uM, less than 20 uM, less than 10 uM, less than 5 uM, less than 1 uM, less than 0.5 uM, less than 0.1 uM, or less than 0.01 uM.

In some embodiments, the peptides of the invention comprise amino acid analogs. In some embodiments, the peptides of the invention comprise the peptides of Family 1, Family 2, Family 3, and/or Family 4 where at least one amino acid of the peptide is substituted with an amino acid analog. Specific examples of amino acid analog substitutions include, but are not limited to, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu, and other aliphatic amino acids; 2-aminoisobutyric acid (Aib) for Gly; cyclohexylalanine (Cha) for Val, Leu and Ile; homoarginine (Har) for Arg and Lys; 2,3-diaminopropionic acid (Dap) for Lys, Arg, and His; N-ethylglycine (EtGly) for Gly, Pro, and Ala; N-ethylglycine (EtGly) for Gly, Pro, and Ala; N-ethylasparagine (EtAsn) for Asn, and Gln; hydroxylysine (Hyl) for Lys; allohydroxylysine (AHyl) for Lys; 3-(and 4-)hydroxyproline (3Hyp, 4Hyp) for Pro, Ser, and Thr; allo-isoleucine (AIle) for Ile, Leu, and Val; 4-amidinophenylalanine for Arg; N-methylglycine (MeGly, sarcosine) for Gly, Pro, and Ala; N-methylisoleucine (MeIle) for Ile; norvaline (Nva) for Met and other aliphatic amino acids; norleucine (Nle) for Met and other aliphatic amino acids; ornithine (Orn) for Lys, Arg and His; citrulline (Cit) and methionine sulfoxide (MSO) for Thr, Asn, and Gln; and N-methylphenylalanine (MePhe), trimethylphenylalanine, halo-(F-, Cl-, Br-, or I-)phenylalanine, or trifluorylphenylalanine for Phe.

More specific examples of compounds of in the invention include an oligonucleotide (which may be an aptamer), antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, the compound may be a closely related protein, for example, a mutated form of Dkk1 or SOST that recognizes LRP5 or LRP6 but imparts no additional effect, thereby competitively inhibiting the action of wild type Dkk1 or SOST. As noted above, the compound, in some embodiments, inhibits the action of Dkk1 or SOST but does not inhibit interactions of Wnt molecules with LRP5 or LPR6.

Additional compounds of the invention include small molecules that interfere with the interaction of Dkk1 with LRP5 and/or LRP6 or the interaction of SOST with LRP5 and/or LRP6. Examples of small molecules include, but are not limited to, peptide-like molecules and synthetic non-peptidyl organic or inorganic compounds.

These small molecules can be identified by any one or more of the screening assays discussed herein and/or by any other screening techniques well known for those skilled in the art.

As described herein, a compound of the invention can be a peptide. Methods of obtaining such peptides are well known in the art, and include screening peptide libraries for binders to a suitable target antigen. In one embodiment, suitable target antigens would comprise LRP5 or LRP6 (or portion thereof that comprises binding site for Dkk1 or SOST), which is described in detail herein. For e.g., a suitable target antigen is the E1 β-propeller of LRP6 or LRP5. Libraries of peptides are well known in the art, and can also be prepared according to art methods. See, for e.g., Clark et al., U.S. Pat. No. 6,121,416. Libraries of peptides fused to a heterologous protein component, such as a phage coat protein, are well known in the art, for e.g., as described in Clark et al., supra. Variants of a first peptide binder can be generated by screening mutants of the peptide to obtain the characteristics of interest (e.g., enhancing target binding affinity, enhanced pharmacokinetics, reduced toxicity, improved therapeutic index, etc.). Mutagenesis techniques are well known in the art. Furthermore, scanning mutagenesis techniques (such as those based on alanine scanning) can be especially helpful to assess structural and/or functional importance of individual amino acid residues within a peptide.

Vector Construction

Polynucleotide sequences encoding the peptides described herein can also be obtained using standard recombinant techniques. Desired polynucleotide sequences may be isolated and sequenced from appropriate source cells. Source cells for antibodies would include antibody producing cells such as hybridoma cells. Alternatively, polynucleotides can be synthesized using nucleotide synthesizer or PCR techniques. Once obtained, sequences encoding the immunoglobulins are inserted into a recombinant vector capable of replicating and expressing heterologous polynucleotides in a host cell. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication (in particular when the vector is inserted into a prokaryotic cell), a selection marker gene, a promoter, a ribosome binding site (RBS), a signal sequence, the heterologous nucleic acid insert and a transcription termination sequence.

In general, plasmid vectors containing replicon and control sequences which are derived from a species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

Either constitutive or inducible promoters can be used in the present invention, in accordance with the needs of a particular situation, which can be ascertained by one skilled in the art. A large number of promoters recognized by a variety of potential host cells are well known. The selected promoter can be operably linked to cistron DNA encoding a polypeptide described herein by removing the promoter from the source DNA via restriction enzyme digestion and inserting the isolated promoter sequence into the vector of choice. Both the native promoter sequence and many heterologous promoters may be used to direct amplification and/or expression of the target genes. However, heterologous promoters are preferred, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In some embodiments, each cistron within a recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

Prokaryotic host cells suitable for expressing polypeptides include Archaebacteria and

Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. Preferably, gram-negative cells are used. Preferably the host cell should secrete minimal amounts of proteolytic enzymes, and additional protease inhibitors may desirably be incorporated in the cell culture.

Polypeptide Production

Host cells are transformed or transfected with the above-described expression vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include Luria broth (LB) plus necessary nutrient supplements. In preferred embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. For example, if a PhoA promoter is used for controlling transcription, the transformed host cells may be cultured in a phosphate-limiting medium for induction. A variety of other inducers may be used, according to the vector construct employed, as is known in the art.

Polypeptides described herein expressed in a microorganism may be secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therefrom. Cells may be removed from the culture and the culture supernatant being filtered and concentrated for further purification of the proteins produced. The expressed polypeptides can be further isolated and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable antigen immobilized on a matrix and Western blot assay.

Besides prokaryotic host cells, eukaryotic host cell systems are also well established in the art. Suitable hosts include mammalian cell lines such as CHO, and insect cells such as those described below.

Polypeptide Purification

Polypeptides that are produced may be purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

Determination of the ability of a candidate substance/molecule compound of the invention to inhibit binding of Dkk1 with LRP5 and/or LRP6 and SOST with LRP5 and/or LRP6, can be performed by testing the modulatory capability of the compound in in vitro or in vivo assays, which are described in the Examples section.

Pharmaceutical Compositions and Modes of Administration

Various compounds (including peptides, etc.) may be employed as therapeutic agents. One embodiment provides pharmaceutical compositions or medicaments containing the compounds of the invention and a therapeutically inert carrier, diluent or excipient, as well as methods of using the compounds of the invention to prepare such compositions and medicaments. In one example, compounds may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound is formulated in an acetate buffer, at pH 5. In another embodiment, the compounds are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular patient being treated, the clinical condition of the individual patient, the cause of the disorder, the site of delivery of the agent, the method of administration, the scheduling of administration, and other factors known to medical practitioners.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Sustained-release preparations may be prepared. Suitable examples of sustained-release preparations include semipermeable matrices of solid hydrophobic polymers containing a compound, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

In one example, the pharmaceutically effective amount of the compound of the invention administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, preferably contain from about 5-100 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 mg, 50 mg, 100 mg, 250 mg or 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound, or a stereoisomer or pharmaceutically acceptable salt thereof In a further embodiment includes a pharmaceutical composition comprising a compound, or a stereoisomer or pharmaceutically acceptable salt thereof, together with a pharmaceutically acceptable carrier or excipient.

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent, or growth-enhancing agent. Such molecules are suitably present in combination in amounts that are effective for the purpose intended.

Screening Methods

In another aspect, the invention provides a method of screening for a compound that inhibits Dkk1 and/or SOST interactions with LRP5 and/or LRP6. The method comprises screening for a compound that binds (preferably, but not necessarily, specifically) to LRP5 and/or LRP6 and inhibits the specific binding of Dkk1 and/or SOST to these receptors.

This invention encompasses methods of screening candidate or test compounds to identify those that inhibit the interactions of Dkk1 with LRP5 and/or LRP6 and compounds that inhibit the interaction of sclerostin with LRP5 and/or LRP6. In one embodiment, the compounds do not inhibit Wnt signaling, Screening assays are designed to identify compounds that bind or complex with LRP5 and/or LRP6, or otherwise interfere with the interaction of LRP5 and/or LRP6 with Dkk1 and/or SOST. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

In one embodiment, the assay calls for contacting the candidate compound with a LRP5 or LRP6 (or equivalent thereof) under conditions and for a time sufficient to allow these two components to interact. In one embodiment, the candidate compound is contacted with the β-propeller domain of E1 of LRP6. In one embodiment, the candidate compound is contacted with the β-propeller domain of E1 of LRP5. In binding assays, the interaction is binding and the complex formed can be isolated or detected in the reaction mixture. In a particular embodiment, a candidate compound is immobilized on a solid phase, e.g., on a microtiter plate, by covalent or non-covalent attachments. Non-covalent attachment generally is accomplished by coating the solid surface with a solution of the substance/molecule and drying. Alternatively, an immobilized affinity molecule, such as an antibody, e.g., a monoclonal antibody, specific for the substance/molecule to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

In other embodiments, interactions between a candidate compound and LRP5 or LRP6, or functionally equivalent portions thereof such as the β-propeller domain of E1 of LRP6 or β-propeller domain of E1 of LRP5, can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Another aspect of the invention provides for an assay that involves using the peptides described herein to screen test compounds for their ability to inhibit interaction of Dkk1 or SOST to an LRP5 or LRP6 target molecule. LRP5 or LRP6 target molecules include the full length LRP5 or LRP6 molecules as well as functionally equivalent portions thereof such as the β-propeller domain of E1 of LRP6 or β-propeller domain of E1 of LRP5. In one embodiment, the assay comprises contacting a test compound with LRP5 or LRP6 target molecule in the presence or absence of a peptide selected from among the peptides of invention, for example a peptide from Family 1, Family 2, Family 3, or Family 4. This peptide is referred to as the peptide ligand in the context of an assay. If the test compound competes for binding with or displaces the peptide ligand from the LRP5 or LPR6 target molecule, then the test compound is selected as a compound that inhibits the interaction of Dkk1 or SOST with the target molecule. The selected test compound can further be evaluated for specific desirable characteristics, such as the ability to promote bone growth, using assays well-known in the art or those described herein, as well as for its effect on the binding of Wnt ligands to the LRP5 or LPR6 target molecule.

The ability of a test compound to inhibit the binding of a peptide ligand to the LPR5 or LRP6 target molecule may be assessed by techniques well known in the art. Either the target molecule, peptide ligand, or test compound can be labeled with a detectable label to facilitate monitoring of assay interactions. Such labels include radioactive isotope, fluorescent labels, chemiluminescent labels, phosphorescent labels, magnetic particles, dyes, metal particles, enzymes, etc. Examples of such labels include, but are not limited to biotin, fluorescein, Texas red, Lucifer yellow, and rhodamine. Other labeling methods include enzymatic tracers, such as alkaline phosphatase, horseradish peroxidase, and glucose oxidase.

Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

A test compound can be any type of molecule, including, for example, a peptide, a peptidomimetic, a peptoid such as vinylogous peptoid, a polynucleotide, or a small organic molecule.

The following are examples of the methods and compositions of the invention. It is understood that various other embodiments may be practiced, given the general description provided above.

All references cited herein, including patent applications and publications, are incorporated by reference in their entirety.

### EXAMPLES

### Example 1

### Materials & Methods

Materials. Highly pure Wnt3a, Wnt9b, Dkk2, Dkk3 and Dkk4 were obtained as carrier-free proteins from R&D Systems (Minneapolis, Minn.) for use in the binding and cell assays. Genens encoding Dkk1, Sclerostin and LRP6 proteins were cloned into a modified pAcGP67 baculovirus DNA transfer vector (BD Pharmingen) for baculovirus generation and extracellular expression in Tni insect cells (Expression Systems, LLC, Woodland Calif.) as previously described (11).

Protein expression and purification. All LRP6, Dkk1 and sclerostin proteins used for this study were expressed and purified according to previously described protocols (11). Pure proteins can be then concentrated to 10 μM stocks and stored at −80° C. The anti-LRP6 E1 YW210.09 Fab was expressed by growing transformed E. coli 34B8 (Stratagene) in low-phosphate AP5 medium at 30° C. for 24 h (43) and was purified over a protein G affinity column (GE Healthcare) (44). Fab-containing fractions were further purified by passage over a SP-Sepharose column (GE Healthcare). Protein concentration was determined by absorbance at 280 nm.

Protein complex crystallization. Purified LRP6 E1E2 was incubated with YW210.09 Fab overnight to form a stable complex, followed by purification of the complex over a Superdex S200 gel-filtration column (GE Healthcare). Fractions containing the complex were pooled and concentrated to 8 mg/mL, then dialyzed into a buffer containing 10 mM Tris pH 8, 300 mM NaCl and 2.5% glycerol. Crystals were obtained from a solution of 0.2M ammonium formate and 20% PEG 3350 (w/v). Because only LRP6E1 was visible in the solved structure, the crystal was analyzed by mass spectrometry, revealing degradation of LRP6 E2 beyond Arg 335. The complex of purified LRP6 E1 and Dkk1 peptide was crystallized from 0.1 M potassium thiocyanate and 30% (w/v) PEG MME 2000 or from 0.2 M NaCl, 0.1 M Tris pH 8, 25% (w/v) PEG 3,350. Crystallization of additional peptides in complex with LRP6 E1 was achieved by micro-seeding the original co-crystals (containing Dkk1 peptide) in the presence of an excess of the peptide of interest (1 to 2 mM final concentration) and LRP6 E1. Seeded crystals grew in 2 or 3 days from one (or both) of the two original Dkk1 peptide crystallization conditions and were found to contain the peptide of interest.

Data collection and structure determination. The diffraction data were collected using a monochromatic X-ray beam (12398.1 eV) at the Advanced Light Source (ALS) beam line 5.0.2. The X-ray detection device was an ADSC quantum-210 CCD detector placed 350 mm away from the crystal. Alternatively, data were collected at Stanford Synchrotron Radiation Laboratory (SSRL71), Advanced Photon Source (APS211DF), or in-house using a Rigaku X-ray generator model 007HF coupled to a Rigaku CCD camera (007HF/Saturn 944+);. Prior to data collection, crystals were transferred into cryo-protective solutions containing 25% glycerol, followed by flash freezing in liquid nitrogen. Rotation method was applied to a single crystal for collection of the complete data set, with 1° oscillation per frame and total wedge size of 180°. The data were then indexed, integrated, and scaled using program HKL2000 (45). The LRP6 E1/Fab structure was phased by the molecular replacement (MR) method using program Phaser (CCP4, Daresbury, England). Matthews' coefficient calculation results indicated that each asymmetric unit was composed of one Fab/E1 complex and 54% solvent. Therefore the MR calculation was directed to search for one set of three subunits including the N-terminal domains of the Fab, the C-terminal domain of the Fab, and the β-propeller domain of E1. The N- and C-terminal domains were searched separately, considering the Fab elbow angle as a variable. The search models of Fab subunits were derived from the crystal structure of an HGFA/Fab complex (46). The search model of the β-propeller domain was a homology model generated through the ESyPred3D web server (47).; the structure of the extracellular domain of LDL receptor (48) was used as the homology modeling template. The difference electron-density map calculated using the MR solution revealed the EGF domain structure. LRP6-peptide complex structures were determined by molecular replacement, using the LRP6E1 domain from the Fab complex as the search model. Peptides were built manually into the electron density. Manual rebuilding was done with the program COOT (49). Structure refinement was carried out with programs REFMAC5 (50) and PHENIX (51) using the maximum likelihood target functions, anisotropic individual B-factor refinement (peptide complexes only.

Binding Assays. Binding kinetics were measured by biolayer interferometry using an Octet Red instrument (ForteBio) as previously described (11). Streptavidin (SA) biosensors were loaded with biotinylated hLRP6 in 50 mM Tris, pH 8, 300 mM NaCl, 5% (v/v) glycerol, and 0.05% (w/v) Triton X-100. The loaded biosensors were washed in the same buffer before carrying out association and dissociation measurements for the indicated times. The K_{d} of each interaction was determined using steady state-analysis through the Octet Red software v6.3. Each reported value represents an average of three or more experiments at different concentrations, with a fitted experimental curve for which the square of the correlation coefficient (R²) is above 0.96.

Alternatively, affinities were determined by fluorescence polarization (FP). A fluorescein-modified peptide probe (30 nM) was mixed with LRP5 or LRP6 E1 domain at a concentration suitable for the affinity of the particular target-probe combination (approximately K_{d}). Competing test agent (protein or peptide) was then added and FP monitored as a function of concentration of the test agent Inhibition constants were obtained by fitting the resulting curves to standard equations using the program KaleidaGraph (Synergy Software).

Peptide affinities were also determined by competition with phage displaying a binding peptide (competition phage ELISA). Serial dilutions of test peptide were mixed with an appropriate (non-saturating) concentration of phage before exposing the mixture to target and allowing it to reach equilibrium. After washing to remove unbound material, bound phage were detected by incubation with anti-M13 antibody-horseradish peroxidase (HRP) conjugate and exposure to a suitable colorometric HRP substrate.

Finally, peptide affinities were also determined by competition ELISA. Maxi-Sorb plates (Nunc) were coated with streptavidin or NeutrAvidin (5 μg/mL in phosphate-buffered saline (PBS); overnight, 4° C.) then blocked with 0.2% bovine serum albumin (BSA) in PBS (1 h, room temperature). A solution (500 nM in PBS) of biotinylated E1-binding peptide Ac-GSLCSNRIKPDTHCSSK(biotin)-am (disulfide) was added to each well for 30 min, and the wells were then washed 3× with PBS containing 0.05% Tween-20 to remove excess peptide. His-tagged E1 domain or FLAG-tagged E1E2 protein (5-10 nM final concentration) was preincubated for 15 minutes with serially diluted test peptide before addition of the mixture to wells of the assay plate for 30 min. Wells were washed and then probed for bound LRP6 by addition of Qiagen penta His-HRP conjugate or Sigma anti-FLAG M2 HRP conjugate (1:2000 dilution in PBS, 0.2% BSA, 0.05% Tween-20) for 30 min. After washing, TMB substrate was added (Kirkegaard and Perry Laboratories). Wells were quenched with 1 M H₃PO₄ and plates read at 450 nm. Inhibition constants were obtained by fitting the resulting curves to standard four-parameter equations using the program KaleidaGraph (Synergy Software).

Light-scattering experiments. Aliquots of 110 μl of protein, or protein complexes equilibrated overnight, were analyzed by SEC-MALS (Dawn Helios 2 with QELS HPLC coupled to Optilab Rex, Wyatt Technologies) as previously described (11).

Phage display. Phage-displayed peptide libraries (approximately 2×10¹⁰ unique members) were constructed as described (41) and cycled through four rounds of solution binding selection against LRP6 E1E2, E1 or E3E4, or against LRP5 E1. Individual phage clones that bound to LRP6 in a phage ELISA were subjected to DNA sequence analysis.

Cellular β-catenin Assay. Wnt signaling was assessed either in mouse fibroblast L-cells or in HEK293s cells. The luciferase reporter assay in 293 cells was performed as described (52). The mouse fibroblast L-cell imaging assay was conducted essentially as described (53). Cells were treated with Wnt3a, Fz8 CRD ((US Patent Publication 20080299136; (54), LRP6, or Dkk1, or combinations of these proteins, as indicated and processed after an additional 6 h at 37° C./5% CO2.

Calvariae bone models. Calvariae are harvested and cultured as previously described (52, 55). Calvariae are cultured in tissue culture plates in BGJb medium supplemented with 0.1% bovine serum albumin and 100 U/ml each of penicillin and streptomycin for 1 day before treating with appropriate concentrations of peptide or protein for 7 days. The bones are cultured in a humidified atmosphere of 5% CO₂ at 37° C. Mouse calvariae are imaged with a μCT 40 (SCANCO Medical, Basserdorf, Switzerland) x-ray micro-CT system. Micro-CT scans are analyzed with Analyze (AnalyzeDirect Inc., Lenexa, Kans., USA). Alternatively, calvariae are stained histologically to view areas of calcification. All experiments using mice are performed in accordance with Genentech Institutional Animal Care and Use Committee guidelines.

### Example 2

### Structure of the LRP6 E1-YW210.09 Fab Complex.

The crystal structure of the first β-propeller and EGF domain of LRP6 (E1 domain) in complex with a Fab from the anti-LRP6 antibody YW210.09 (W02011119661) was determined by molecular replacement and refined to 1.9 Å resolution with an R and R free of 0.175 and 0.220 respectively. The crystallographic asymmetric unit is composed of one LRP6 E1 domain and one YW210.09 Fab. Interpretable electron density allowed tracing of the residues Ala20 to Lys324 for the E1 domain. With the exception of Fab heavy chain residues Ser127 to Thr131, residues Asp1 to Glu213 and Glu1 to Lys214 could be traced for the Fab light chain and heavy chain, respectively. (Kabat numbering is used throughout (56)).

The LRP6 E1 domain is assembled in a modular architecture that comprises a β-propeller module and an epidermal growth factor (EGF) like module. The β-propeller consists of six blades formed by a four-stranded anti-parallel β-sheet arranged radially, with the N-terminal edge facing the center channel and the YWTD motifs located in the second strand of each blade. The LRP6 E1 β-propeller structure closely resembles that of LDLr (57) with an rmsd of 0.83 Å when superimposed over 245 Cα atoms, despite a sequence identity of only 36%. Most of the conserved residues are concentrated around the YWTD core motifs forming the β-sheets, essential to the β-propeller structural integrity. In contrast, the surface residues are highly diverse, as might be expected from the functional diversity of these receptors. LRP6 uses its EGF-like domain to lock down the first and sixth blades of the propeller (to maintain its mechanical strength). The EGF-like module extends out C-terminally from the β-propeller via a ten-residue linker and then folds back on to the bottom side of β-propeller, docking to a surface between the third and fourth blades. The interaction between EGF-like domain and β-propeller is extensive, as indicated by the large total buried surface area of 1226 Å² and a shape complimentarity score of 0.74 (58). Three residues in the first β-strand of the EGF module, Leu296, Leu298 and Met299, constitute a hydrophobic core that packs into a complementary cavity of the β-propeller; the hydrophobic core is surrounded by a number of direct or water-mediated polar interactions. These features are also observed in LDLr structures (48, 57).

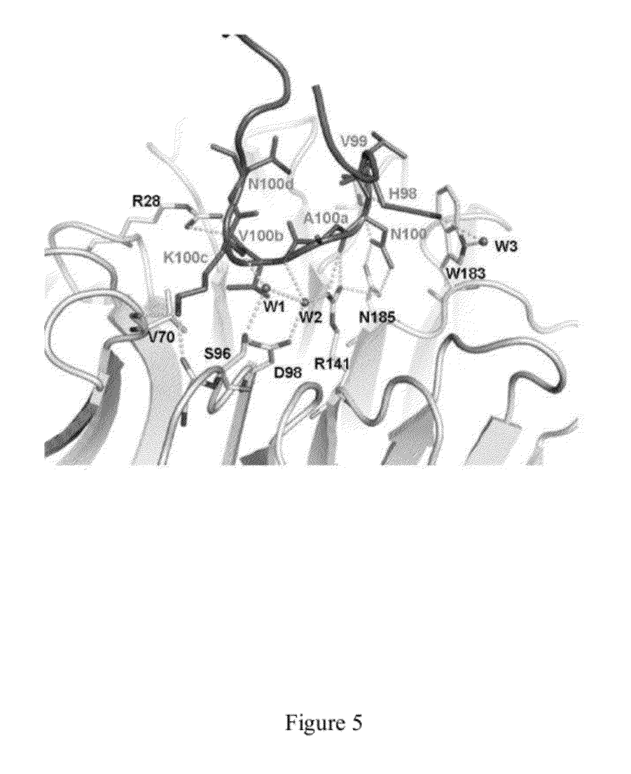

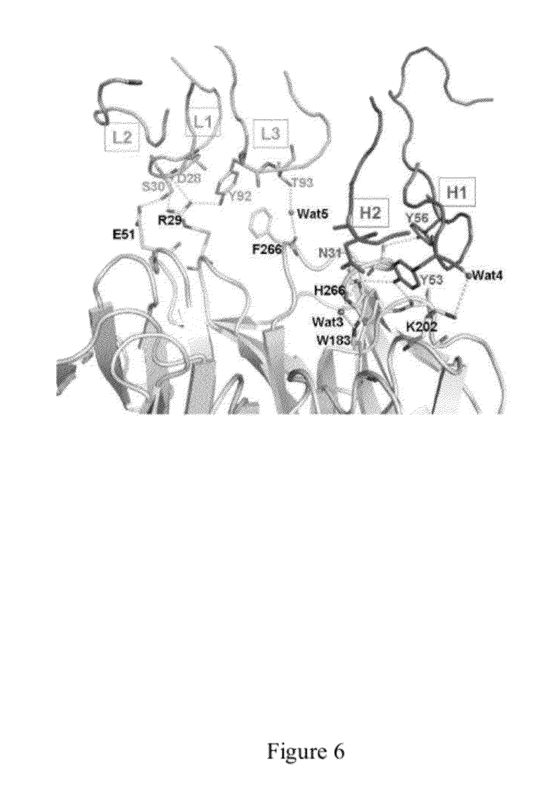

YW210.09 Fab recognizes a region at the top center of the β-propeller, an area that is frequently found to be involved in protein-protein interactions (59). The paratope is composed of residues from five of the CDRs, including three heavy chain CDRs (H1, H2, H3) and two light chain CDRs (L1 and L3). Antibody binding to the β-propeller buries a total area of 1691 Å², with a shape complementarity score of 0.76. An acidic patch occupies roughly a third of the total surface area on this side of the β-propeller but barely overlaps with the YW210 epitope. Antibody heavy chain and light chain recognize discrete areas. Direct contacts formed by the heavy chain CDRs represent 80% of the buried surface area, with CDR H3 alone accounting for over 50%. This segment is composed of 17 residues, among which residues His98 to Lys100c form direct contacts with the β-propeller. Importantly, Asn100 of the antibody makes a pair of hydrogen bonds with Asn185 of LRP6, forming a “hand shake” interaction (FIG. 5). In addition, the unusual main chain conformation through Val100b and Lys100c positions a carbonyl group that interacts with Arg28 of LRP6 in the “back”, and two NH groups which interact with the acidic patch through two water molecules (Wat1 and Wat2) in the “front” (FIG. 5). The Lys100c side chain also neutralizes in part the acidic patch by hydrogen bonding with Val70 and Ser96 main chain carbonyls of LRP6. Arg141 of LRP6 is anchored in the middle and interacts with the bridging water Wat2, Asn185 of LRP6, and Ala100a of YW210.09. Arg141 appears to integrate the two hydrogen-bond networks. Additionally, the Val100b side chain docks into a hydrophobic cavity in the center channel of the β-propeller. Therefore, the short, contiguous YW210.09 H3 sequence NAVK exhibits an unusually significant degree of interaction with the β-propeller E1 of LRP6. The other CDRs interact with residues along the perimeter of the top of the β-propeller. H1 and H2 contact the fifth and sixth blades, while L1 and L3 contact the sixth, the first, and the second blades (FIG. 6). Crystal packing interactions are not directly involved in the areas where the YW210.09 contacts the LRP6 epitope, indicating that the crystal structure should reflect how the two molecules interact in solution.

### Example 3

### YW210.09 H3 Loop Sequence Presents an “NXI” Motif Conserved Among Dkks, Sclerostin and Wise.

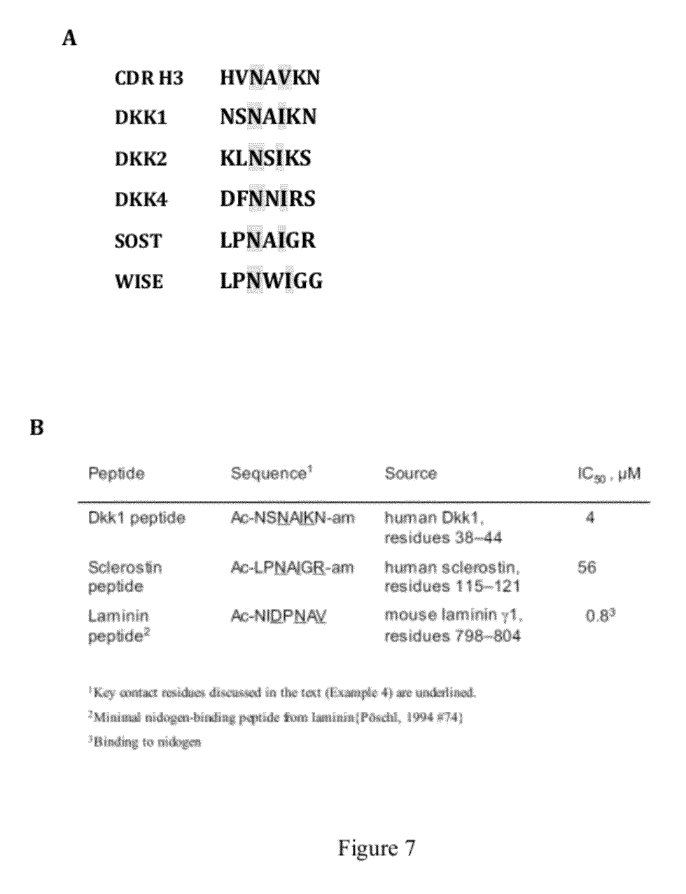

The interaction between the distinct CDR H3 NAVKN motif and LRP6 E1 β-propeller is highly similar to the interaction reported between laminin and nidogen (60). In both cases, significant contacts are made through the Asn handshake described above and a branched hydrophobic residue entering a hydrophobic cavity formed by the top of β-propeller center channel. In contrast to LDLr, the center of the nidogen and LRP6 E1 channels is closed off from solvent by a tryptophan residue held in place by a nearby phenylalanine side chain, or “Phe shutter” (60). This feature has been proposed to be predictive of YWTD propeller domains that can bind to low molecular-weight ligands (60). A short sequence of human Dkk1 (NAIKN; amino acids 40 to 44) is nearly identical to the motif found in the CDR H3 loop of YW210.09 (FIG. 7). This motif is strictly conserved among multiple Dkk family members from different species, with the exception of Dkk3. Strong conservation suggests that this segment of Dkks1, 2, and 4 has an important function. The conserved motif is found near the N-terminus of Dkk1, a region which is predicted to be disordered and which has not been identified previously as functionally important (61). Additionally, this motif appears in two other proteins regulating Wnt signaling via interaction with LRP5/6, namely sclerostin (32) and wise (30) (FIG. 7A). Sclerostin and wise belong to the super-family of cystine-knot proteins (62) and display the motif in the extended loop 2, also called the “heel” of this well-defined fold (63, 64). In the case of sclerostin, the“heel” has been mapped as the binding epitope for a neutralizing antibody (63), suggesting that the region may be functionally important. No details of the sclerostin or wise interaction with LRP5 or LRP6 have been reported.

### Example 4

Peptides from Dkk1 and Sclerostin Bind to the Top of the LRP6 β-Propeller.

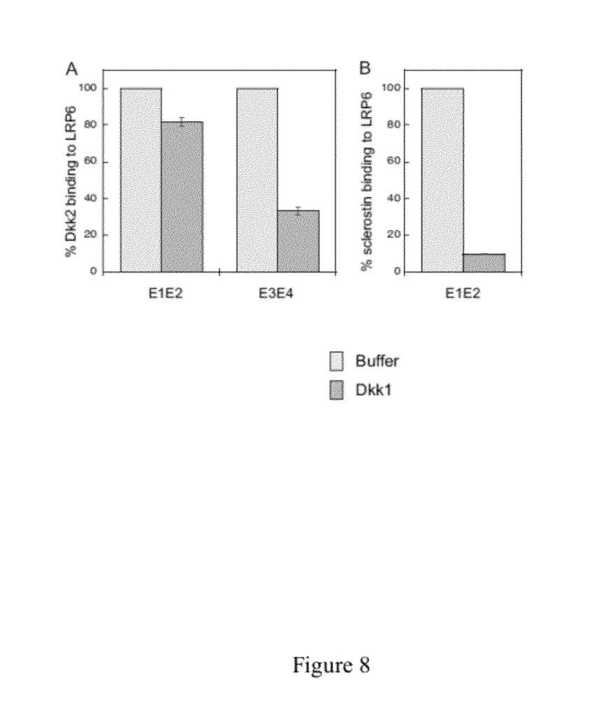

Seven-residue peptides from Dkk1 and Sost were synthesized by standard Fmoc procedures. FIG. 7B. These peptides include the “NXI” motif described above. Affinities of the peptides for LRP6 E1E2 were determined by competition phage ELISA (41). The Dkk1 peptide binds with relatively high affinity, while the sclerostin peptide binds about 10-fold more weakly (IC₅₀s 4 μM and 45 μM, respectively) These values are comparable to the affinity of a laminin peptide for the nidogen β-propeller (65). To understand the detailed interactions of the peptides with LRP6, we determined high-resolution co-crystal structures of the peptides bound to the LRP6 E1 β-propeller. Structures were determined by molecular replacement and refined to 1.9 and 1.5 Å resolution for Dkk1 and Sost peptides, respectively (FIG. 8). Remarkably, the peptides show very similar bound-state conformations compared to the antibody loop, in each case placing the key asparagine side chain in position for the “handshake” interaction described above. Peptide isoleucine residues occupy the hydrophobic pocket where the valine side chain of the antibody loop interacts. The overall comparison of the antibody loop and the Dkk1 peptide is especially striking; alignment of antibody residues Val99 to Lys100c (backbones Cα-to-Cα, including also the Lys β-carbon and the entire side chains of Asn100, Ala100a, and Val100b) with the equivalent atoms of the Dkk1 peptide shows that the conformations are essentially identical (RMSD of 0.14 Å over 26 atoms). In addition to the core “NXI” motif, basic side chains in each peptide interact with the acidic patch on LRP6, despite their different relative positions in the sequence. For the Dkk1 peptide, lysine immediately follows the isoleucine residue; the ε-amino group of lysine occupies a small acidic cleft in a manner very similar to the interaction of the analogous lysine from the antibody loop. In the case of the Sost peptide, the isoleucine is followed by an intervening glycine before the basic arginine residue. This reorients the peptide backbone and places the arginine side chain in a more peripheral location on the acidic patch of LRP6. These peptide structures demonstrate that binding to LRP6 E1 is driven by both an extremely well-defined core motif (interactions of the Asn and Ile side chains) and by interactions with a surrounding surface capable of a range of supporting contacts. This latter group of interactions is likely responsible not only for additional affinity but also for specificity. For example, a related peptide from laminin requires Asn and Val residues for high-affinity binding to nidogen (65), and these form very similar contacts to those seen for the “NXI” motif in the LRP6 complex structures (60). However, high-affinity interaction with nidogen requires an additional contact from an Asp that occurs two residues before the Asn of the core motif (60, 65). This Asp forms a salt bridge with a surface Arg that is present in nidogen but not in LRP5 or LRP6. Overall, the binding properties of the Dkk1 and sclerostin peptides are consistent with the idea that the “NXI” motif observed in multiple Wnt pathway inhibitors (FIG. 7) is important for the binding of these proteins to LRP5 and LRP6 and, therefore, for their inhibitory activity.

### Example 5

### Mapping of Interactions Between Wnt Pathway Inhibitors and the Individual β-Propellers of LRP6.

The interaction of the different Dkks and sclerostin with various domains of LRP6 was measured using a biolayer interferometry assay (11). Purified receptors contained individual β-propeller-EGF-like units (E1, E2, or E4), two β-propellers (E1E2 or E3E4), or four β-propellers (E1E4))—as follows:

Human LRP6: construct E1E4—amino acids A20-Q1253 of LPR6; construct E1E2—amino acids A20-E631 of LPR6; construct E3E4 amino acids E631-Q1253 of LPR6; construct E1—amino acids A20-D325 of LPR6; construct E2—amino acids D235-E631 of LPR6; construct E4-T933-Q1253. The individual LRP6 β-propeller E3 could not be expressed.

Human LRP5: construct E1—amino acids P33-R348 of LRP5

Dkk1 can bind to both the E1E2 and the E3E4 regions of LRP6 (11). This study extends that finding by showing that both Dkk1 and Dkk2 bound to LRP6 E1E2 with high affinity (22 and 53 nM, respectively). Furthermore, Dkk1 and Dkk2 also bound to E3E4 (51 and 38 nM, respectively). In contrast, high-affinity interactions were not observed for Dkk3 and Dkk4. Dkk3 failed to bind to any LRP6 construct tested, in agreement with a recent report (66). Dkk4 showed some evidence of very weak binding to LRP6 E1E4 and E3E4 but, interestingly, did not bind to E1E2. Further analysis of Dkk1 and Dkk2 binding to the individual β-propellers indicates that they each bind with high affinity only to E1. Binding to E4 was undetectable, indicating that the observed interaction with E3E4 is likely driven by a high-affinity interaction with E3. Partial binding to E2 was detectable only at very high Dkk concentrations, consistent with either very weak binding or with a non-specific effect. Binding of sclerostin was mapped in a similar manner. Sclerostin binds only to E1E4, E1E2, and E1, with only very weak or non-specific binding to E2.

To assess whether Dkk1, Dkk2, and sclerostin might bind to the same site on LRP6 E1, Dkk2 or sclerostin binding was measured in the presence of preloaded Dkk1 (100 nM). Dkk2 binding was inhibited only slightly (FIG. 8A), suggesting that the binding sites for Dkk1 and Dkk2 do not significantly overlap. In contrast, sclerostin binding is very strongly inhibited in the presence of Dkk1 (FIG. 8B), suggesting overlapping binding sites for these two inhibitors. This conclusion is consistent with the peptide interaction studies above showing that the “NXI” motifs of Dkk1 and sclerostin interact with LRP6 E1 in a similar manner.

### Example 6

### The “NXI” Motif is Important for Binding of Dkk1 and Sclerostin to LRP6 E1.

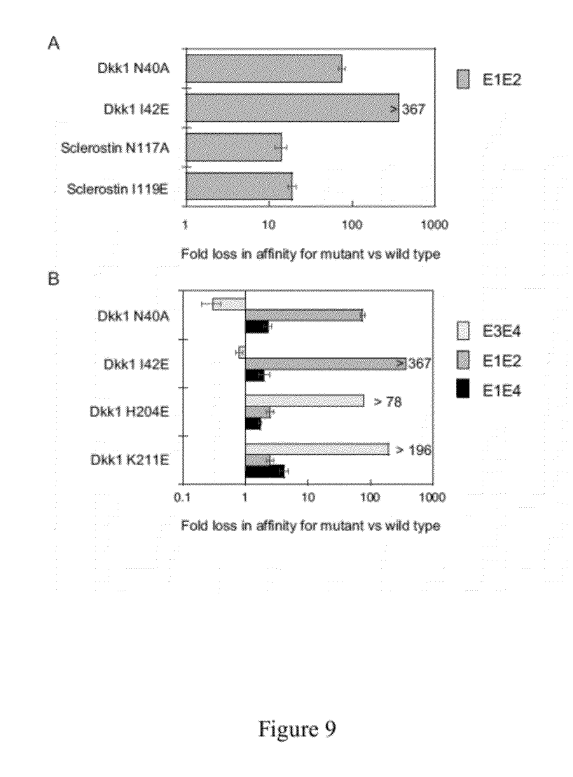

Based on the combined results of the peptide and domain mapping studies, it was hypothesized that binding of Dkk1 and sclerostin to LRP6 E1 is mediated primarily by the “NXI” motif present in each protein. To test this idea, the key contact residues in the motif were substituted with amino acids predicted to disrupt the interaction (Asn-to-Ala; or Ile-to-Glu). Notably, substitutions of the analogous residues in laminin dramatically impact binding to nidogen, with a losses in affinity of 3000- to 50,000-fold (67). The Asn40Ala substitution in Dkk1 resulted in a 75-fold loss in affinity for LRP6 E1E2, while the Ile42Glu substitution largely abolished binding (>364-fold effect) (FIG. 9A). The impact of the substitutions on sclerostin binding is clearly evident but not as strong, with 14- and 19-fold losses in affinity for the Asn117Ala and Ile119Glu substitutions, respectively (FIG. 9A). These data are consistent with an important role for the “NXI” motif, especially for Dkk1.

### Example 7

### Amino Acid Substitutions in CRD2 of Dkk1 Disrupt Binding to LRP6 E3E4.

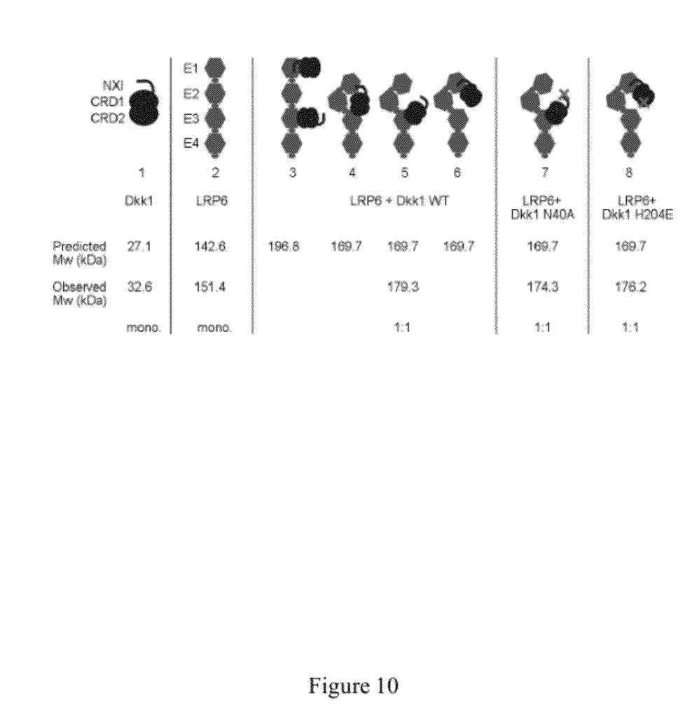

An important role for the C-terminal region of Dkk proteins has been proposed; for example, it has been shown that mRNAs encoding human Dkk1 or Dkk2 lacking the first cysteine-rich domain (CRD1) can inhibit xWnt8 signaling when injected into Xenopus embryos (61). To date, however, there is no complete structure available of any Dkk family member, nor of any complex with an interaction partner. An experimental structure of CRD2 from mouse Dkk2 has been computationally docked onto a homology model of LRP5 E3 (68). Based on this model of the complex, substitution of mouse Dkk1 residues His210, Lys217 or Arg242 (corresponding to human Dkk1 residues 204, 211, and 236, respectively) with Glu was predicted to interfere with binding (68) and, in each case, was found to disrupt both binding to cells transfected with LRP6 and the ability of Dkk1 to inhibit Wnt3a signaling (69). As described in Example 5, Dkk1 can bind to both the E1 and, presumably, the E3 domains of LRP6. We therefore suspected that the affinity for LRP6 E3 might be much lower for human Dkk1 incorporating the reported amino acid substitutions in CRD2 and that binding to E1 would be unaffected. This hypothesis was tested with Dkk1 mutants H204E and K211E, with the results shown in FIG. 9B. Indeed, these mutations interfered with binding to LRP6 E3E4 but not to E1E2. In agreement with results described above, the converse was true for “NXI” motif substitutions. Interestingly, neither CRD2 nor “NXI” motif substitutions had more than a slight effect on Dkk1 binding to E1E4. Taken together, this suggests that that Dkk1 binds independently to two different sites on E1E4 (2:1 complex; cartoon 3 in FIG. 10), or alternatively, that Dkk1 can bind to two sites that are mutually exclusive (i.e., alternative 1:1 complexes; cartoons 5 and 6 in FIG. 10). A third possibility is that a single Dkk1 molecule binds to sites on E1 and E3 simultaneously (cartoon 4 in FIG. 10); however the lack of any substantial “avidity effect” for wild-type Dkk1 compared to the two classes of mutants would appear to make this less likely. In addition, no formation of a ternary complex of E1E2, Dkk1, and E3E4 was observed (11). To distinguish 2:1 and 1:1 binding models, complexes of Dkk1 variants with E1E4 were analyzed by size-exclusion chromatography coupled with light-scattering detection. These data show that all of the Dkk1 E1E4 complexes with LRP6 E1E4 exhibit 1:1 stoichiometry (FIG. 10). Overall, the affinity measurements and light-scattering data suggest the existence of two independent, but mutually exclusive, binding modes between Dkk1 and LRP6.

### Example 8

### Sclerostin Regulates Only a Subset of Wnts Whereas Dkk1 Act as a Broad Inhibitor of the Pathway.

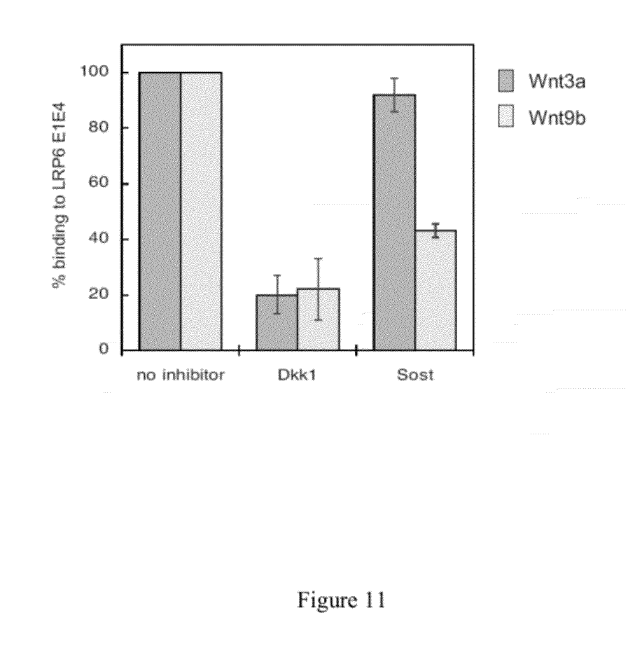

As described above (Example 5), sclerostin binds to LRP6 E1 and does not interact with the E3E4 region of LRP6. Wnt9b also binds to the E1E2 region but not to the E3E4 region (11). Accordingly, sclerostin inhibits Wnt9b binding to LRP6 E1E4 (FIG. 11). In contrast, sclerostin is unable to inhibit the binding of Wnt3a to LRP6 E1E4 (FIG. 11), in agreement with previous observations that Wnt3a does not bind to E1E2 but instead binds to the E3E4 region of LRP6 (11). The situation with Dkk1 is more complex, as Dkk1 can bind with high affinity to both E1E2 and E3E4 fragments of LRP6 (11), apparently through two distinct modes of interaction (Examples 5-7). Accordingly, Dkk1 inhibits the binding of both Wnt3a and Wnt9b to LRP6 E1E4 (FIG. 11).

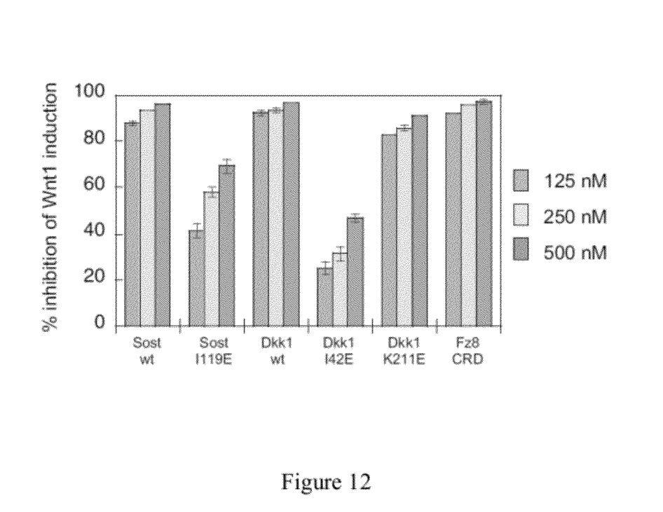

To test whether the observed effects on binding between purified proteins were relevant to cellular signaling, Dkk1 and sclerostin activities were tested further in a Wnt-dependent TOPbrite luciferase reporter assay (52). Cells stably transfected with reporter were transfected transiently with Wnt1. The Wnt-transfected cells were treated with purified Dkk1 or sclerostin variants, and the effect on reporter induction was measured (FIG. 12). For wild-type Dkk1 and sclerostin, strong inhibition is observed of Wnt1-dependent signaling. This is consistent with earlier observations that Wnt1 belongs to a class of Wnts signaling through the E1E2 portion of LRP5/6 (11, 52). The Dkk1 and sclerostin mutants show activities consistent with their binding to LRP6 (FIG. 12). Sclerostin Ile119Glu (“NXI” motif) is impaired relative to wild-type in its ability to inhibit Wnt1-driven signaling, as is Dkk1 Ile42Glu. In contrast, Dkk1 Lys211Glu (CRD2) efficiently inhibits Wnt1 signaling, consistent with the retained ability of this mutant to bind to E1E2 (FIG. 9B).

Taken together, the binding data and the effects on Wnt signaling in the cellular assay confirm that the conserved “NXI” motif is functionally relevant for Dkk1 and sclerostin inhibition of those Wnts signaling through binding to E1E2, and that the Dkk1 CRD2 interaction with E3E4 is important only for inhibition of a different subset of Wnt ligands. In addition, the data show that Dkk1 inhibits Wnt signaling broadly (through two distinct binding modes), while sclerostin is more selective.

### Example 9

Human Bone Mineral Density (BMD) Mutations Disrupt Dkk1 and Sclerostin Binding to LRP6 E1E2 without Affecting Wnt9b Binding.

Understanding the Dkk1 and sclerostin interaction with the first β-propeller of LRP6 sheds light on the mechanism of LRP5 gain-of-function mutations. These single amino-acid substitutions in LRP5 E1 lead to significant increases in bone strength and thickness in affected individuals (22-24). Over the last eight years, a total of nine LRP5 gain-of-function mutations (at seven positions) have been described (23). Each of these seven amino acids is strictly conserved between LRP5 and LRP6. Overall, the E1 β-propellers of LRP5 and LRP6 are highly conserved (68% identical), and, significantly, their top interacting surfaces are almost entirely identical. From a structural point of view, the most striking of the BMD mutations is the substitution of Asn198 with Ser (24); Asn198 corresponds to LRP6 Asn185 that is engaged in the “handshake” interaction with the “NXI” motif found in Dkk1 and sclerostin (Examples 2 and 4). Mapping the sites of BMD mutations on the surface of the LRP6 E1/Dkk1 peptide complex revealed that in addition to Asn185, LRP6 residues Asp98, Arg141, and Ala201, either make direct contacts with the peptide or are immediately adjacent to the binding pocket. Accordingly, these mutations can be predicted to disrupt Dkk1 and sclerostin binding to LRP6 E1.

The other three sites of mutation are more distant from the bound peptide, but the substitutions might be expected to have indirect effects on the integrity of the peptide binding pocket. LRP6 residue Gy158 is present on a surface loop and might be expected to influence the conformation of Trp157. The indole ring of Trp157 sits next to the BMD mutation site Arg141, where it may screen the hydrogen bond between the Arg side chain and the carbonyl group of the peptide Asn from solvent. The indole of Trp157 also makes up one wall of the pocket surrounding the Asn-Asn “handshake”. Adjacent to the Gly158 loop is a second indole side chain, that of Trp183; this indole forms a second wall of the Asn-Asn pocket. Ala201, another BMD mutation site, is on a surface loop on the other side of Trp183 from Gly158. Incorrect positioning of the side chains of Trp157 or Trp183 would be expected to disrupt binding of the “NXI” peptide. BMD sites Thr240 and Ala229 are positioned away from the surface of the protein near the ends of adjacent β-strands. Thr240 occurs in one of the characteristic “YWTD” repeats present in this class of propeller proteins. The Thr240 hydroxyl group hydrogen bonds to the backbone amide of Ala229; substitution at either residue might be expected to cause destabilization of the protein. In addition, Ala229 lies immediately under the “Phe shutter” (see Example 3) thought to be important for closing off the bottom of the ligand binding site from solvent (60).

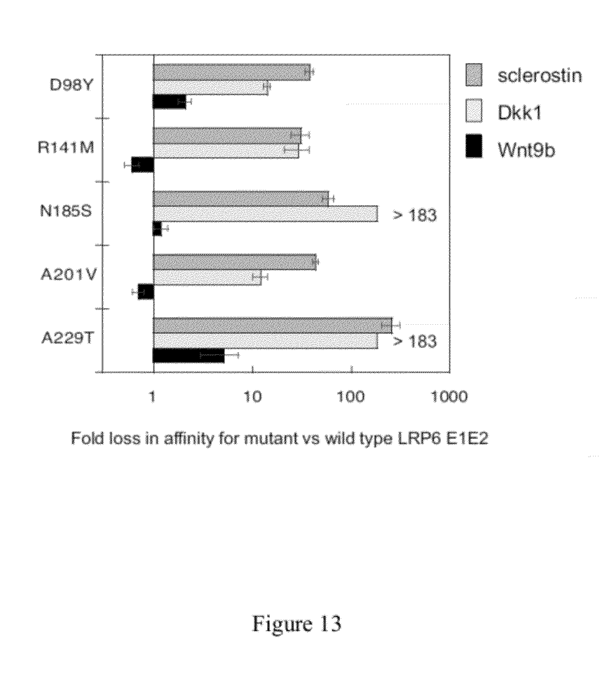

Cells transfected with LRP5 variants carrying BMD mutations show reduced binding to sclerostin and are less sensitive to sclerostin inhibition of Wnt10b or Wnt6 signaling (70). To further test the effects of the BMD substitutions, we introduced several of them into LRP6 E1E2, with the results shown in FIG. 13. LRP6 E1E2 Gly158Val could not be expressed in insect cells. This observation is in line with the extremely low levels of expression observed in mammalian cells for the corresponding LRP5 mutant (70), suggesting that substitution of this residue is structurally destabilizing. All of the other LRP6 mutations we tested disrupt the binding of both Dkk1 and sclerostin to LRP6 E1E2. Mutation of Asn185 to Ser significantly disrupts binding with a losses in affinity of 183- and 59-fold for Dkk1 and sclerostin, respectively. Similarly, Arg141Met induces losses in affinity of 29- and 31-fold for Dkk1 and sclerostin, respectively. Importantly, there is little to no effect on the binding of Wnt9b binding to the LRP6 variants. These results support the idea that the gain of function resulting from BMD mutations does not result from a gain in affinity for Wnt ligands, but instead from a selective loss in affinity for Wnt inhibitors. Importantly, not only is the binding to sclerostin affected (70), but the binding of Dkk1 to its E1 interaction site is also impaired.

### Example 10

LRP5 and LRP6 E1 β-Propellers are Highly Specific Peptide Recognition Modules.

The LPR6 β-propellers were probed for peptide binding specificity using phage display. Naive libraries of linear or cyclic peptides were used for solution binding experiments against LRP6 E1E2 or E3E4, or LRP5 E1 (41). Each target was used to perform four rounds of binding selection. A dramatic enrichment was observed for binding to specific target over binding to BSA, with 1000- and 6000-fold enrichment for LRP6 E1E2 and E3E4, respectively. Similar strong enrichment was observed for selection against LRP5 E1 domain. Individual phage clones were screened for binding to the target of interest and also for binding to other LRP6 β-propeller constructs. Phage selected against LRP6 E1E2 or E3E4 constructs were remarkably specific: all isolated clones bound only to the original target with no cross-binding to other LRP6 constructs. In addition, phage specific for E1E2 bound only to the E1 domain, while phage selected against E3E4 appear to be specific for E3. Sequences of peptides were obtained from sequencing phage clones of interest. Particularly promising clones were used to design secondary libraries for affinity maturation; these libraries were subjected to additional rounds of selection and screening.

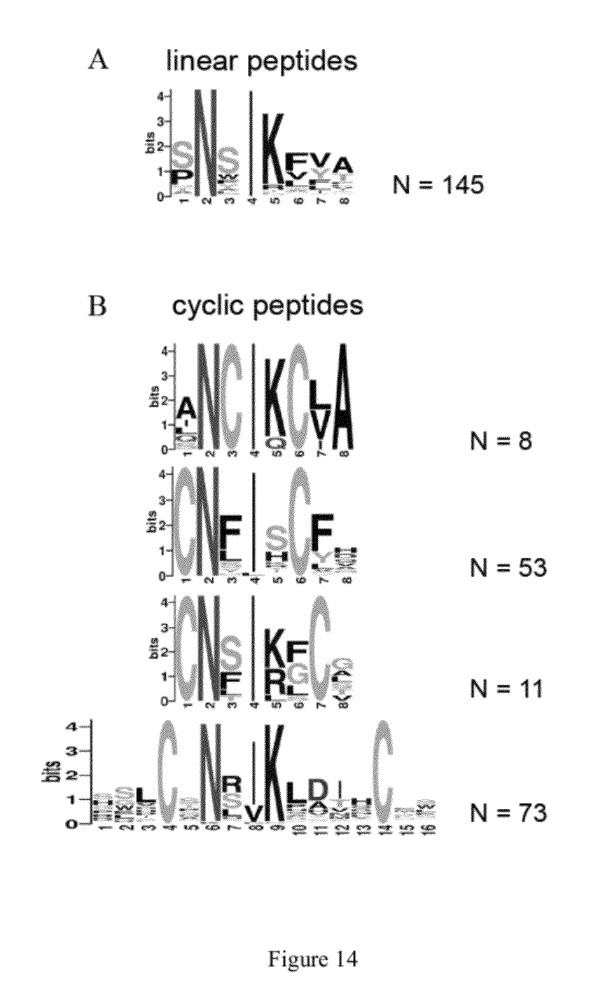

LRP6 E1 peptide sequence motifs (FIG. 14) are remarkably consistent with the “NXI” motif found in Dkk1, sclerostin and wise. For both linear and cyclic peptides libraries, a strictly conserved Asn is present (position 0). At position +2, there is invariably a branched hydrophobic residue, with Ile being present in the overwhelming majority of cases. The strong selection for these residues in specifically binding phage confirms the importance of these two residues in the “NXI” motif. For peptides derived from linear libraries, a residue that could render a turn, such as Pro, Ser, Cys or Gly, is preferred at the −1 position. Ser is the most preferred for the +1 position, followed by hydrophobic residues such as Phe, Trp, Tyr and Leu. As observed in the Dkk1 sequence, Lys is the most preferred residue at position +3, with Arg and His as the second and third most common residues. Finally, hydrophobic residues are preferred at position +4 and +5. Cyclic libraries that included a wide range of loop lengths between the two cysteines yielded, after binding selection, cyclic peptides of only four types. These differ both in loop length and in the position of the “NXI” motif relative to the Cys residues. In addition, residue preferences at positions flanking the conserved Asn and Ile residues are different for different cycle types. For example, the preference for Lys at +3 is considerably relaxed for cycles of the type “CNXIXC”. In other cases, for example cycles of the type “CXNXIKX₄C”, the underlined Lys is nearly invariant. These results are consistent not only with a strong specificity for the “NXI” motif, but also with distinct conformational preferences (and potentially binding contacts) for the different types of cyclic peptides.

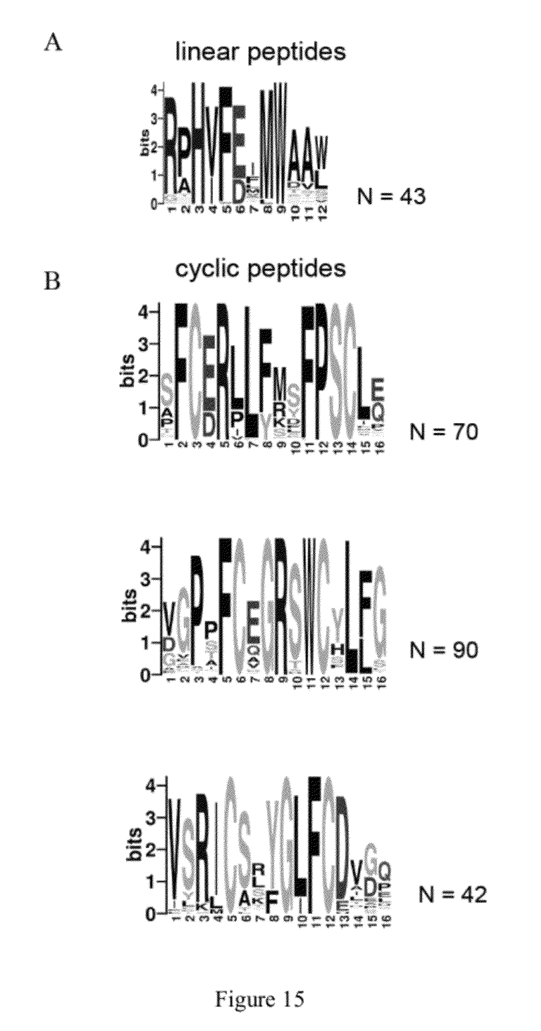

Peptides binding to LRP5 E1 were obtained in the same manner as described above for LRP6 E1E2. Like LRP6, LRP5 yielded distinct linear and cyclic peptide motifs (FIG. 15). However, these motifs were rather different from those binding to LRP6 E1. In particular, these peptides do not contain the “NXI” motif. The linear peptides instead show a conserved acidic position (position 0), with hydrophobic amino acids at positions +2, +3 and −1, (Met, Trp, and Phe, respectively). Matured clones show a very strong preference for His at −3 and Arg at −5. Two of the three cyclic peptide families also have a conserved acidic residue, but their sequence patterns are otherwise distinct from that of the linear family.

### Example 11

Synthesis of Peptides Identified from Phage Library Selections Confirms that they Bind to LRP6 E1.

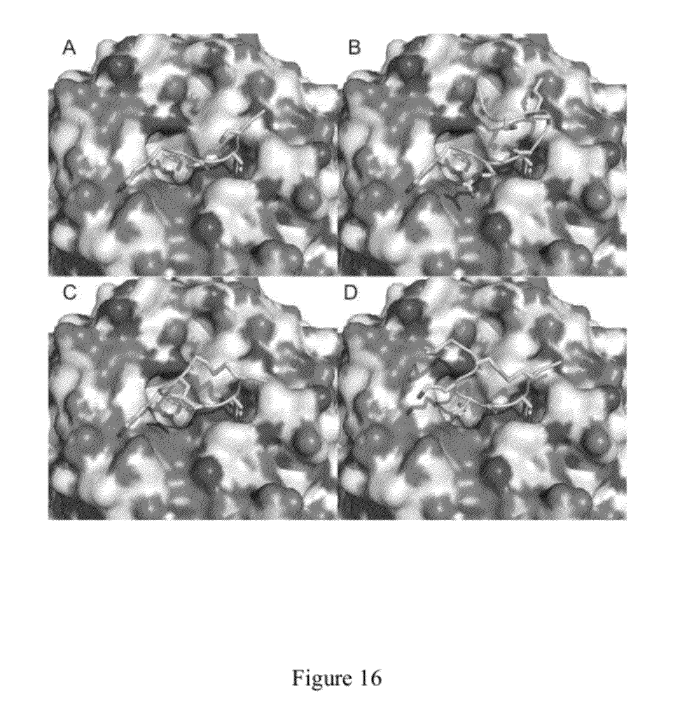

Several peptides from Exemplary Families 1 and 2 were chemically synthesized to assess whether they bound to target (LRP6 E1) outside of the context of display on phage particles. In general, these synthetic peptides were capable of binding to target. Affinities for the linear peptides of Exemplary Family 1 were in the same range as the Dkk1 7-mer peptide (low micromolar), while cyclic peptides from Exemplary Family 2 had affinities of low micromolar to mid-nanomolar. To understand how these phage-derived peptides recognized LRP6 E1, several co-crystal structures were determined (FIG. 16). The four peptides in the structures shown all contain “NXI” motifs; accordingly, all four peptides bind to the same site as Dkk1 and sclerostin peptides and the Asn and Ile residues of each peptide occupy the same sites described above for other structures. In addition, the peptide structures show some unique features. The “CX₉C” cyclic peptide shown in part B places an N-terminal acetyl group into a third shallow pocket on the surface of LRP6 (top center). This pocket is not occupied by the Dkk1 peptide. Interestingly, several residues of this peptide (those after the Lys shown toward botton left) are not visible in the electron density, suggesting that they are dynamic in the bound state. The peptides in the structures shown in parts C and D are closely related in sequence, differing only in reversal of the last two residues. This residue reversal has the additional effect of contracting the cycle size from “CX₅C” to “CX₄C”. It can be seen that the two peptides make slightly different contacts with the protein; in particular, the Lys side chain interaction is different, and, accordingly, peptide affinity is affected.

### Example 12

### Determination of the Minimal Binding Sequence of the Dkk1 Peptide and Substitution of Individual Residues in a Minimized Analogue.

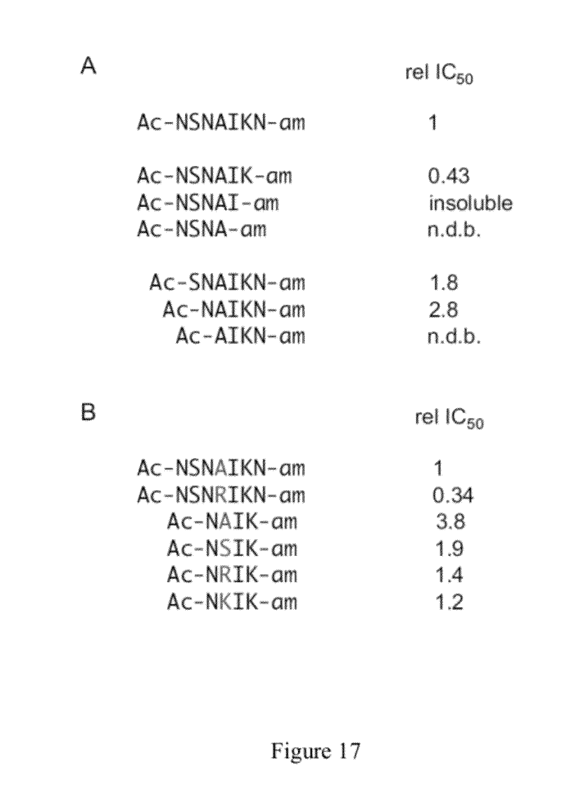

To determine whether all seven residues of the Dkk1 peptide were necessary for binding to LRP6 E1, several shorter peptides were synthesized. These peptides lacked one or more residues taken from the N-terminus or from the C-terminus. Removal of three residues from either end completely abolished binding. These deletions were sufficient to remove either the conserved Asn or the conserved Ile of the “NXI” motif. This confirms the importance of both of these residues for binding to the LRP6 site. Lesser deletions generally preserved binding, with effects on affinity of no more than 3-fold. FIG. 17(A and B).

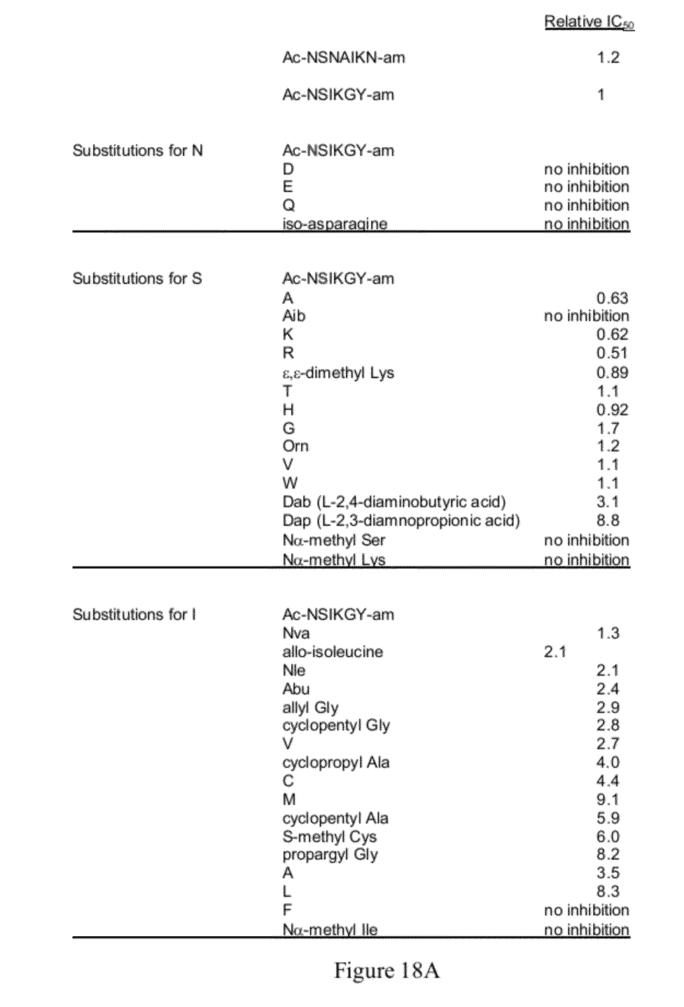

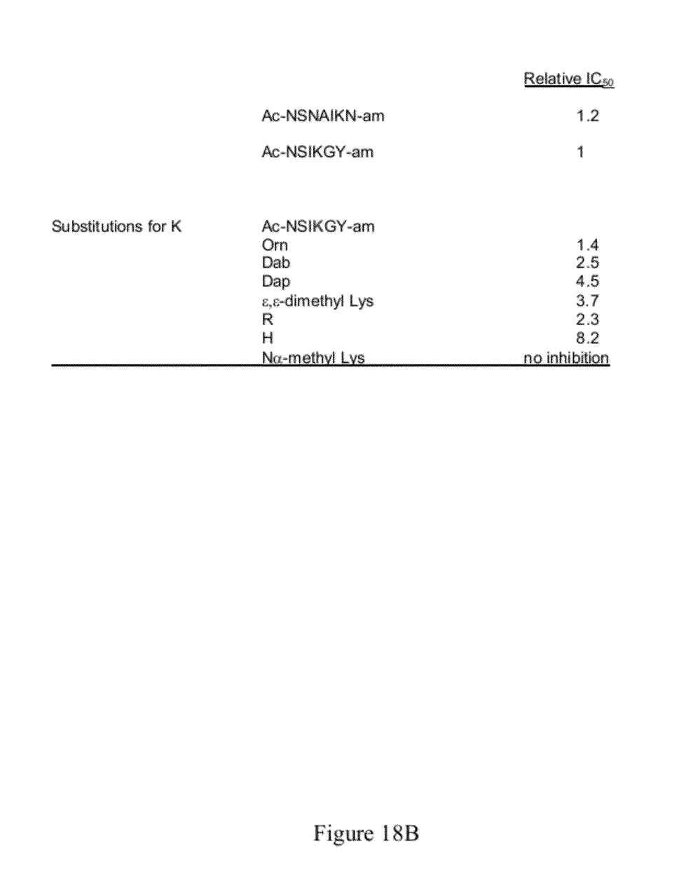

Results from a substitution study are shown in FIG. 18. Substitutions of the Asn residue in the peptide Ac-NSIKGY-am confirmed the importance of this residue in binding to LRP6 E1 domain. In particular, the normally conservative substitution Gln resulted in complete loss of detectable binding. Substitutions of the S, I, and K residues were generally more tolerated. Replacement of Ser with Ala or with basic residues Lys, Arg, His, or ε,ε-dimethyl Lys slightly improved affinity, although basic residues with shorter side chains, such as Orn, Dab, and Dap showed lower affinity as the length of the side chain decreased. Many hydrophobic substitutions for Ile were tolerated, although amino acids with larger side chains, such as Phe, were not. Relatively long side chains such as those of Leu or Met caused significant loss of affinity. The β-methyl group of Ile appears to have minimal importance, as Nva bound with affinity similar to the Ile-containing parent, and a similar pattern was also observed for Val and Abu analogues. However, substitution of the Ile residue with a charged residue (Glu) abolished binding. Finally, the Lys residue could be replaced by a variety of basic amino acids. Of these, Orn and Arg peptides retained affinity close to that of the Lys parent, while amino acids with shorter side chains (Dap and Dab) reduced peptide affinity. Substitution of Na-methyl amino acids at any position tested (S, I, or K) caused substantial loss of affinity (binding not detected).

### Example 13

Further Exploration of the Hydrophobic Pocket.

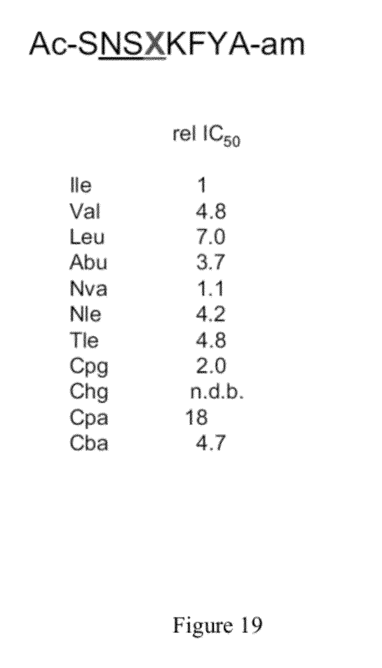

The preference for side chains at the Ile position of the “NXI” motif was explored in the context of a peptide from Exemplary Family 1 (the parent peptide is the same as that shown in FIG. 16A). Ten additional peptides were synthesized, each with a different hydrophobic amino acid in place of the Ile residue (Table 2; FIG. 19). With the exception of the cyclohexylglycine (Chg) substitution, each of the peptides bound to LRP6. The peptide incorporating the non-genetically encoded amino acid norvaline (Nva) was equipotent to the parent Ile peptide. In addition, the Tle peptide bearing three β-methyl groups was equipotent with the Val peptide (bearing two such methyl groups). From both of these comparisons, it can be inferred that LRP6 E1 can accommodate one extra β-methyl group on the peptide side chain (or a loss of such a methyl group) with no deleterious effect on affinity.

### Example 14

Transfer of the “NXI” Motif to a Structured Peptide Scaffold.

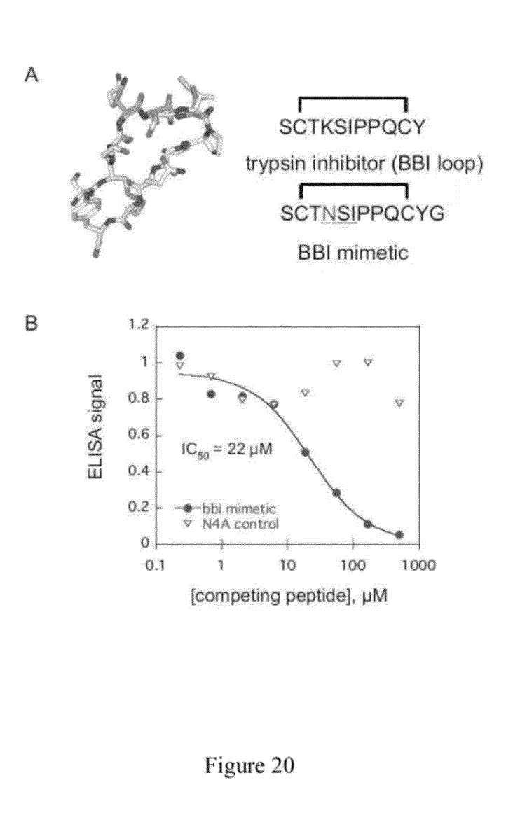

A peptide having both the side chains necessary to make specific contacts and a well-defined (and appropriate) conformation in solution might be expected to exhibit higher affinity for a target protein. With this idea in mind, the structure of ligands bound to LRP6 was compared to published peptide structures. The unusual backbone conformation around the Asn of the “NXI” motif appeared matched to a class of plant protease inhibitors (Bowman-Birk inhibitors; BBI). Short, disulfide-bonded inhibitory peptides can be taken from the larger, natural inhibitors, and structures of some of these peptides have been determined (42). Comparing over several residues, the backbone conformation of the “NXI” motif overlaid closely with the BBI peptide structure (FIG. 20A). The only significant sequence difference was a BBI loop Lys (the P1 determinant for trypsin inhibition) compared to the Asn required for binding to LRP6. Synthesis of a BBI-related peptide with this single amino acid change yielded a peptide with affinity for LRP6 (22 μM; FIG. 20B). Notably, this BBI mimetic has no equivalent to the Lys residue in Dkk1 that interacts with the acidic patch of LRP6. This shows that the “NXI” motif is sufficient to bind to LRP6 E1.

### Example 15

Peptide Cyclization Strategies Other than Disulfide Bonds.

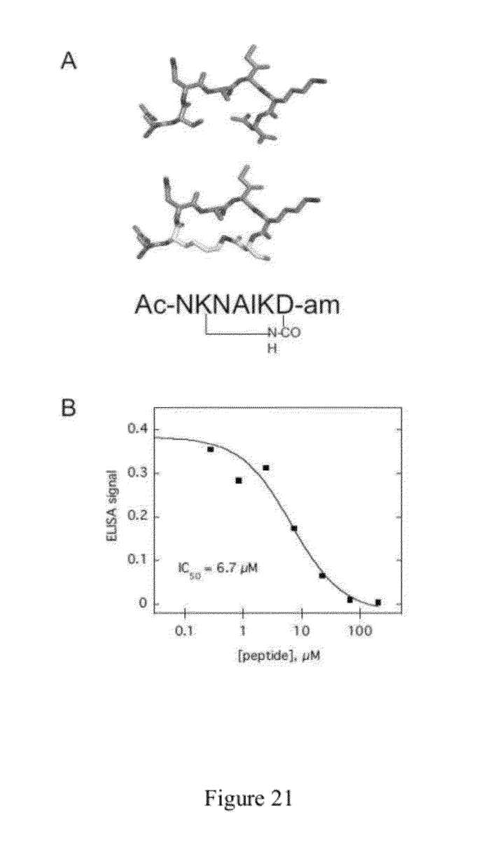

As described in the preceding Example, cyclization of peptides can improve affinity for a target protein. In addition, cyclization may, in some cases, enhance stability in biological settings or otherwise improve the properties of a peptide for use in modulating a biological effect. It is therefore of interest to define a variety of cyclization methods for a given peptide. The structure of the Dkk1 peptide bound to LRP6 suggested such a strategy (FIG. 21A). The bound peptide is bent in a way that places sidechains of the second (Ser) and seventh (Asn) residues pointing toward one another. The distance is such that it might be joined by amide bond formation between a Lys side chain (in place of the Ser) and an Asp side chain (in place of the Asn). The target cyclic peptide was synthesized and found to bind to LRP6 with affinity equivalent to the parent Dkk1 peptide (FIG. 21B).

### Example 16

“NXI” Motif Peptides Inhibit Binding of Wnt Inhibitors to LRP6 but do not Inhibit Wnt Binding.

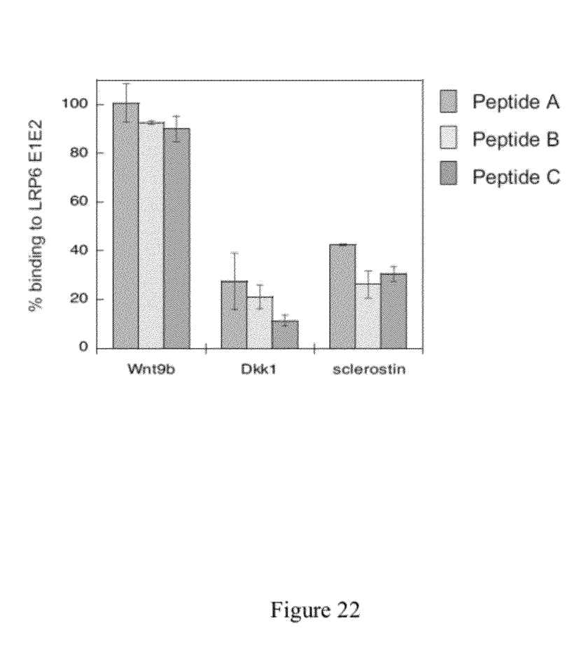

A therapeutic strategy directed at stimulation or restoration of Wnt-stimulated bone growth might be most effective if the action of inhibitors can be eliminated without interference with positive signaling by Wnt ligands. The possibility that inhibitor binding and Wnt binding might be separable (because of distinct epitopes on LRP5/6) was suggested by experiments with BMD mutant analogues of LRP6 (Example 9). To support the conclusions from protein mutagenesis, peptides were assayed for inhibitory activity toward binding of various ligands to LRP6. Three different peptides inhibited binding of the inhibitors Dkk1 and sclerostin to LRP6 E1E2 without affecting the binding of Wnt9B (FIG. 22). This shows that low molecular-weight ligands can recapitulate the effect of BMD mutations.

### Example 17

### Compounds can be Tested in an ex vivo Bone Growth Assay.

To assess whether a peptide or other agent might have useful effects on bone growth, an ex vivo bone growth assay is used. This assay follows the development of the skulls (calvaria) of mouse embryos in culture. The developing bone produces a number of relevant cell types, for example osteoblasts, and the dissected calvaria are sufficiently complex to respond to treatments in a manner indicative of potential in vivo responses. In addition, the calvaria assay is more convenient than treatment of an animal. In general, calvaria are harvested and split into halves for assay as previously described (see Example 1) (52, 55). Peptides are dissolved in water at 50 times the target assay concentration then diluted fresh daily into assay medium. The medium is changed daily for 7 days. At the end of this growth period, samples are processed for analysis as described (52, 55). Histological staining (alizarin red/alcian blue) reveals areas of calcification in red.

### REFERENCES

1. Clevers H. 2006. Cell 127: 469-80
2. MacDonald B T, Tamai K, He X. 2009. Dev Cell 17: 9-26
3. Nusse R. 2008. Cell Res 18: 523-7
4. Polakis P. 2007. Curr Opin Genet Dev 17: 45-51
5. Nusse R, Varmus H E. 1982. Cell 31: 99-109
6. Nusse R, Brown A, Papkoff J, Scambler P, Shackleford G, et al. 1991. Cell 64: 231
7. Rijsewijk F, Schuermann M, Wagenaar E, Parren P, Weigel D, Nusse R. 1987. Cell 50: 649-57
8. Bhanot P, Brink M, Samos C H, Hsieh J C, Wang Y, et al. 1996. Nature 382: 225-30
9. Pinson K I, Brennan J, Monkley S, Avery B J, Skarnes W C. 2000. Nature 407: 535-8
10. Tamai K, Semenov M, Kato Y, Spokony R, Liu C, et al. 2000. Nature 407: 530-5
11. Bourhis E, Tam C, Franke Y, Bazan J F, Ernst J, et al. 2010. J Biol Chem 285: 9172-9
12. Noordermeer J, Klingensmith J, Perrimon N, Nusse R. 1994. Nature 367: 80-3
13. Tamai K, Zeng X, Liu C, Zhang X, Harada Y, et al. 2004. Mol Cell 13: 149-56
14. Zeng X, Huang H, Tamai K, Zhang X, Harada Y, et al. 2008. Development 135: 367-75
15. Zeng X, Tamai K, Doble B, Li S, Huang H, et al. 2005. Nature 438: 873-7
16. Mani A, Radhakrishnan J, Wang H, Mani M A, Nelson-Williams C, et al. 2007. Science 315: 1278-82
17. Caricasole A, Copani A, Caraci F, Aronica E, Rozemuller A J, et al. 2004. J Neurosci 24: 6021-7
18. De Ferrari G V, Papassotiropoulos A, Biechele T, Wavrant De-Vrieze F, Avila M E, et al. 2007. Proc Natl Acad Sci USA 104: 9434-9
19. Kinzler K W, Nilbert M C, Vogelstein B, Bryan T M, Levy D B, et al. 1991. Science 251: 1366-70
20. Nishisho I, Nakamura Y, Miyoshi Y, Miki Y, Ando H, et al. 1991. Science 253: 665-9
21. Gong Y, Slee R B, Fukai N, Rawadi G, Roman-Roman S, et al. 2001. Cell 107: 513-23
22. Boyden L M, Mao J, Belsky J, Mitzner L, Farhi A, et al. 2002. N Engl J Med 346: 1513-21
23. Rickels M R, Zhang X, Mumm S, Whyte M P. 2005. J Bone Miner Res 20: 878-85
24. Whyte M P, Reims W H, Mumm S 2004. N Engl J Med 350: 2096-9; author reply-9
25. Hoang B, Moos M, Jr., Vukicevic S, Luyten F P. 1996. J Biol Chem 271: 26131-7
26. Dann C E, Hsieh J C, Rattner A, Sharma D, Nathans J, Leahy D J. 2001. Nature 412: 86-90
27. Hsieh J C, Kodjabachian L, Rebbert M L, Rattner A, Smallwood P M, et al. 1999. Nature 398: 431-6
28. Glinka A, Wu W, Delius H, Monaghan A P, Blumenstock C, Niehrs C. 1998. Nature 391: 357-62
29. Semenov M V, Tamai K, Brott B K, Kuhl M, Sokol S, He X. 2001. Curr Biol 11: 951-61
30. Itasaki N, Jones C M, Mercurio S, Rowe A, Domingos P M, et al. 2003. Development 130: 4295-305
31. Li X, Zhang Y, Kang H, Liu W, Liu P, et al. 2005. J Biol Chem 280: 19883-7
32. Semenov M, Tamai K, He X. 2005. J Biol Chem 280: 26770-5
33. Bafico A, Liu G, Yaniv A, Gazit A, Aaronson S A. 2001. Nat Cell Biol 3: 683-6
34. Balemans W, Ebeling M, Patel N, Van Hul E, Olson P, et al. 2001. Hum Mol Genet 10: 537-43
35. Balemans W, Patel N, Ebeling M, Van Hul E, Wuyts W, et al. 2002. J Med Genet 39: 91-7
36. Li X, Liu P, Liu W, Maye P, Zhang J, et al. 2005. Nat Genet 37: 945-52
37. Rawadi G. 2008. Curr Drug Targets 9: 581-90
38. Roux S. 2010. Joint Bone Spine
39. Bennett C N, Longo K A, Wright W S, Suva L J, Lane T F, et al. 2005. Proc Natl Acad Sci USA 102: 3324-9
40. Walsh N C, Gravallese E M. 2010. Immunol Rev 233: 301-12
41. Tonikian R, Zhang Y, Boone C, Sidhu S S. 2007. Nat Protoc 2: 1368-86
42. Brauer A B, Kelly G, McBride J D, Cooke R M, Matthews S J, Leatherbarrow R J. 2001. J Mol Biol 306: 799-807
43. Lee C V, Liang W C, Dennis M S, Eigenbrot C, Sidhu S S, Fuh G. 2004. J Mol Biol 340: 1073-93
44. Fellouse F A, Esaki K, Birtalan S, Raptis D, Cancasci V J, et al. 2007. J Mol Biol 373: 924-40
47. Lambert C, Leonard N, De Bolle X, Depiereux E. 2002. Bioinformatics 18: 1250-6
48. Rudenko G, Henry L, Henderson K, Ichtchenko K, Brown M S, et al. 2002. Science 298: 2353-8
49. Emsley P, Cowtan K. 2004. Acta Crystallogr D Biol Crystallogr 60: 2126-32
50. Murshudov G N, Vagin A A, Dodson E J. 1997. Acta Cryst D53: 240-55
51. Adams P D, Afonine P V, Bunkoczi G, Chen V B, Davis I W, et al. 2010. Acta Crystallogr D Biol Crystallogr 66: 213-21
52. Gong Y, Bourhis E, Chiu C, Stawicki S, DeAlmeida V I, et al. 2010. PLoS ONE 5: e12682
53. Hannoush R N. 2008. PLoS One 3: e3498
54. DeAlmeida V I, Miao L, Ernst J A, Koeppen H, Polakis P, Rubinfeld B. 2007. Cancer Res 67: 5371-9
55. Mohammad K S, Chirgwin J M, Guise T A. 2008. Methods Mol Biol 455: 37-50
56. Wu T T, Kabat E A. 1970. J Exp Med 132: 211-50
57. Jeon H, Meng W, Takagi J, Eck M J, Springer T A, Blacklow S C. 2001. Nat Struct Biol 8: 499-504
58. Lawrence M C, Colman P M. 1993. J Mol Biol 234: 946-50
59. Springer T A. 1998. J Mol Biol 283: 837-62
60. Takagi J, Yang Y, Liu J H, Wang J H, Springer T A. 2003. Nature 424: 969-74
61. Brott B K, Sokol S Y. 2002. Mol Cell Biol 22: 6100-10
62. McDonald N Q, Hendrickson W A. 1993. Cell 73: 421-4
63. Lintern K B, Guidato S, Rowe A, Saldanha J W, Itasaki N. 2009. J Biol Chem 284: 23159-68
64. Veverka V, Henry A J, Slocombe P M, Ventom A, Mulloy B, et al. 2009. J Biol Chem 284: 10890-900
65. Poschl E, Fox J W, Block D, Mayer U, Timpl R. 1994. EMBO J 13: 3741-7
66. Nakamura R E, Hackam A S. Growth Factors
67. Poschl E, Mayer U, Stetefeld J, Baumgartner R, Holak T A, et al. 1996. EMBO J 15: 5154-9
68. Chen L, Wang K, Shao Y, Huang J, Li X, et al. 2008. J Biol Chem 283: 23364-70
69. Wang K, Zhang Y, Li X, Chen L, Wang H, et al. 2008. J Biol Chem 283: 23371-5
70. Semenov M V, He X. 2006. J Biol Chem 281: 38276-84
71. Bourhis, E. et al., 2011. Structure, 19: 1433-1442

1. An isolated peptide comprising the amino acid sequence X₀X₁X₂X₃ where X₀ is N; X₁ is A, S, F, T, Y, L, or K, or R; X₂ is I or V; and X₃ is K, R, or H.

2. The peptide of claim 1, wherein the peptide comprises the amino acid sequence X₁X₀X₁X₂X₃X_{4,} where X₋₁ is P, S, C, or G; X₀ is N; X₁ is A, S, F, T, Y, L, or K, or R; X₂ is I or V; X₃ is K, R, or H; and X₄ is F, T, Y, L, or V.

3. The peptide of claim 1, wherein the peptide comprises an amino acid sequence selected from the group consisting of N X₁IK, N X₁VK, N X₁ IR, N X₁ VR, N X₁ IH, and N X₁VH, where X₁ is A, S, F, T, Y.

4. The peptide of claim 1, wherein the peptide is selected from the group consisting of the peptides of Family 1 (FIG. 1).

5. The peptide of claim 4, wherein at least one amino acid of the peptide is substituted with an amino acid analog.

6. The peptide of claim 1, wherein the peptide comprises an amino acid analog.

7. The peptide of claim 1, wherein the peptide inhibits the binding of Dkk1 to LRP6 and does not inhibit the binding of Wnt9B to LRP6.

8. The peptide of claim 1, wherein the peptide binds to the E1 β-propeller of LRP6.

9. The peptide of claim 8, wherein the peptide interacts with at at least one, at least two, at least three, at least four, at least five, at least six, at least seven, at least eight, at least nine, at least ten, at least eleven, or all of the amino acid residues R28, E51, D52, V70, S71, E73, L95, S96, D98, E115, R141, and N185 of the E1 β-propeller of LRP6.

10. An isolated cyclic peptide comprising the amino acid sequence: X₀X₁X₂X_{3,} where X₀ is N; X₁ is F, Y, L, A, R, or S; X₂ is I or V; and X₃ is K, R, or H.

11. The cyclic peptide of claim 10, wherein the cyclic peptide comprises the amino acid sequence X₋₁X₀X₁X₂X₃X_{4,} where X₋₁ is P, S, C, or G; X₀ is N; X₁ is F, Y, L, A, R, or S; X₂ is I or V; X₃ is K, R, or H; and X₄ is F, T, Y, L, or V.

12. The cyclic peptide of claim 10, wherein the cyclic peptide comprises an amino acid sequence from the group consisting of N X₁IK, N X₁VK, N X₁ IR, N X₁ VR, N X₁ IH, and N X₁VH, where X₁ is F, Y, L, A, R, or S.

13. The cyclic peptide of claim 10, wherein the peptide is selected from the group consisting of the peptides of Family 2 (FIG. 2).

14. The cyclic peptide of claim 13, wherein at least one amino acid of the peptide is substituted with an amino acid analog.

15. The cyclic peptide of claim 10, wherein the peptide comprises an amino acid analog.

16. The cyclic peptide of claim 10, wherein the peptide inhibits the binding of Dkk1 to LRP6 and does not inhibit the binding of Wnt9B to LRP6.

17. The cyclic peptide of claim 10, wherein the peptide binds to the E1 β-propeller of LRP6.

18. The cyclic peptide of claim 10, wherein the peptide interacts with at at least one, at least two, at least three, at least four, at least five, at least six, at least seven, at least eight, at least nine, at least ten, at least eleven, or all of the amino acid residues R28, E51, D52, V70, S71, E73, L95, S96, D98, E115, R141, and N185 of the E1 β-propeller of LRP6.

19. An isolated peptide comprising the amino acid sequence: X₋₁X₀X₁X_{2,} where X₋₁ is W, L, Y, F, or I; X₀ is D or E; X₁ is F, W, I, S, or Y; and X₂ is M.

20. The peptide of claim 19, wherein the peptide comprises the amino acid sequence: X₋₂X₋₁X₀X₁X₂X_{3,} where X₋₂ is V, I, L, or F; X₋₁ is W, L, Y, F, or I; X₀ is D or E; X₁ is F, W, I, S, or Y; X₂ is M; and X₃ is W, M, A, or G.

21. The peptide of claim 19, wherein the peptide is selected from the group consisting of the peptides of Family 3 (FIG. 3).

22. An isolated peptide selected from the group consisting of the peptides of Family 4 (FIG. 4).

23. A method for screening for a compound that inhibits the interaction of Dkk1 and LRP6 comprising
contacting a test compound with LRP6, or functional equivalent thereof, and
determining the level of binding of the test compound to the LRP6, or functional equivalent thereof, in the presence and the absence of a peptide ligand that inhibits the interaction of Dkk1 with LRP6,
wherein a change in level of binding in the presence or absence of the peptide ligand indicates that the test compound inhibits the interaction of Dkk1 with LRP6,
and wherein the peptide ligand comprises an amino acid sequence selected from the group consisting of the amino acid sequences of
a) Family 1 (FIG. 1);
b) Family 2 (FIG. 2);
c) Family 3 (FIG. 3); and
d) Family 4 (FIG. 4).

24. The method of claim 23, wherein the peptide ligand is labeled with a detectable label.

25. A method for screening for a compound that inhibits the interaction of Dkk1 and LRP5 comprising
contacting a test compound with LRP5, or functional equivalent thereof, and
determining the level of binding of the test compound to the LRP5, or functional equivalent thereof, in the presence and the absence of a peptide ligand that inhibits the interaction of Dkk1 with LRP5,
wherein a change in level of binding in the presence or absence of the peptide ligand indicates that the test compound inhibits the interaction of Dkk1 with LRP5
and wherein the peptide ligand comprises an amino acid sequence selected from the group consisting of the amino acid sequences of
a) Family 1 (FIG. 1);
b) Family 2 (FIG. 2);
c) Family 3 (FIG. 3); and
d) Family 4 (FIG. 4).

26. The method of claim 25, wherein the peptide ligand is labeled with a detectable label.
